(19) United States
(12) Patent Application Publication
(10) Pub. No.: US 2025/0388678 A1
(43) Pub. Date: Dec. 25, 2025

(54) Methods, Combinations, and Uses for the Treatment of Muscle Invasive Bladder Cancer

(71) Applicant: AstraZeneca AB, Sodertalje (SE)

(72) Inventors: Ashok Gupta, Gaithersburg, MD (US); Salvatore Ferro, Gaithersburg, MD (US)

(21) Appl. No.: 19/247,786

(22) Filed: Jun. 24, 2025

Related U.S. Application Data
(60) Provisional application No. 63/664,153, filed on Jun. 25, 2024.
(60) Provisional application No. 63/722,332, filed on Nov. 19, 2024.
(60) Provisional application No. 63/756,569, filed on Feb. 10, 2025.

(51) Int. Cl. C07K 16/28; A61K 31/555; A61K 31/7068; A61K 39/00; A61P 35/00

(52) U.S. Cl. CPC C07K 16/2827; A61K 31/555; A61K 31/7068; A61P 35/00; A61K 2039/505; A61K 2039/545

(57) ABSTRACT

The disclosure relates to methods for treating patients with muscle-invasive bladder cancer using durvalumab and platinum-based chemotherapy. The present disclosure also relates to combination therapies comprising durvalumab and platinum-based chemotherapy for the treatment of patients with muscle invasive bladder cancer. The present disclosure further relates to the use of combination therapies comprising durvalumab and platinum-based chemotherapy for the manufacture of a medicament for treating patients with muscle invasive bladder cancer.

### FIELD OF THE DISCLOSURE

The present disclosure relates to methods for treating patients with muscle invasive bladder cancer using durvalumab and platinum-based chemotherapy. The present disclosure also relates to combination therapies comprising durvalumab and platinum-based chemotherapy for the treatment of patients with muscle invasive bladder cancer. The present disclosure further relates to the use of combination therapies comprising durvalumab and platinum-based chemotherapy for the manufacture of a medicament for treating patients with muscle invasive bladder cancer.

### SEQUENCE LISTING

This application contains a sequence listing which is submitted electronically and is hereby incorporated by reference in its entirety. The sequence listing submitted herewith is contained in the XML filed created Jun. 25, 2024 entitled “24-0716-US—PRO_Sequence-Listing.xml” and is 7,789 bytes in size.

### BACKGROUND OF THE DISCLOSURE

Bladder cancer (BC) is the 9th most common cancer worldwide, with an estimated 429,800 new cases diagnosed each year and more than 165,000 deaths reported globally in 2012 (Ferlay et al., 2015; Torre et al., 2015). Bladder cancer is generally divided into muscle-invasive (MIBC) and non-muscle-invasive disease (NMIBC) based on invasion of the muscularis propria. At the initial diagnosis of BC, 70% to 75% of cases are diagnosed as NMIBC, and approximately 25% to 30% are diagnosed as MIBC (Babjuk et al., 2014; Boccardo et al., 2006; Burger et al., 2013).

Cisplatin-based neoadjuvant chemotherapy is the standard first-line (1 L) treatment in patients with MIBC fit enough to receive chemotherapy. The combination of neoadjuvant therapy and radical cystectomy has shown potential for an increase in pathologic complete response (pCR), event-free survival (EFS), and overall survival (OS) in patients with MIBC versus radical cystectomy alone (Grossman et al., 2003; Sonpavde et al., 2009). Consistent with this, the treatment recommendation for MIBC is cisplatin-based neoadjuvant chemotherapy with radical cystectomy surgery for patients with stage II and stage IIIa MIBC (AJCC 8th edition).

Despite this, recurrence rates of bladder cancer leading to death remain high. Programmed cell death-1 (PD-1) and programmed cell death-ligand 1 (PD-L1) have demonstrated activity in patients with metastatic BC and have been granted approval in the platinum refractory setting. The benefits of adjuvant chemotherapy after radical cystectomy in patients with MIBC without clinically detectable metastases are unclear. However, meta-analyses (Leow et al., 2014; Kim et al., 2017) suggest an OS benefit in patients receiving adjuvant cisplatin-based chemotherapy while use of adjuvant PD-1 or PD-L1 inhibitors reveal promising activity and safety (Necchi et al., 2018; Powles et al., 2018). Durvalumab is a human monoclonal antibody (mAb) of the immunoglobulin (Ig) G1 kappa (IgG1κ) subclass that inhibits binding of PD-L1 (B7 homolog 1 [B7-H1], cluster of differentiation [CD]274) to PD-1 (CD279) and CD80 (B7-1). Nonclinical and clinical studies have indicated that blockade of the immune checkpoints PD-1/PD-L1 can have a positive effect on antitumor activity. Despite this, MIBC patients have high rates of disease recurrence with possible development of advanced cancer, with most recurrences occurring within the first 2 to 3 years after cystectomy (Chang et al., 2017; Witjes et al., 2016).

Therefore, there is still a significant unmet medical need for additional treatment options to improve survival in this patient population. Thus, new therapies and protocols are needed to further improve the long-term prognosis for bladder cancer patients.

### SUMMARY OF THE DISCLOSURE

The disclosure demonstrates that treatment of patients with bladder cancer with perioperative durvalumab plus neoadjuvant platinum-based chemotherapy results in significant improvement of their clinical response (e.g., EFS, OS and pCR) as compared to patients treated with neoadjuvant platinum-based chemotherapy only. Thus, the disclosure generally relates to methods for treating bladder cancer, including muscle invasive bladder cancer (MIBC). In one aspect, the disclosure provides a method of treating bladder cancer in a patient in need thereof, the method comprising: (a) administering to the patient an anti-PD-L1 antibody or antigen binding fragment thereof and one or more platinum-based chemotherapies; and (b) performing a radical cystectomy.

In another aspect, the disclosure provides a method of treating bladder cancer in a patient in need thereof, the method comprising: (a) administering to the patient about 1500 mg of durvalumab and one or more platinum-based chemotherapies about every 3 weeks (Q3W) for about 4 cycles; (b) performing a radical cystectomy; and administering to the patient an adjuvant therapy comprising about 1500 mg of durvalumab about every 4 weeks (Q4W) for about 8 cycles.

In another aspect, the disclosure provides a method of treating bladder cancer in a patient in need thereof, the method comprising: (a) administering to the patient: (i) about 1500 mg of durvalumab; (ii) gemcitabine at a dose of about 1000 mg/m² on days 1 and 8 of each cycle; (iii) cisplatin at a dose of about 70 mg/m² on day 1 of each cycle if the patient has a creatinine clearance of about ≥60 mL/min or at a dose of about 35 mg/m² on days 1 and 8 of each cycle if the patient has a creatinine clearance of about ≥40 mL/min to <60 mL/min; wherein the durvalumab, gemcitabine, and cisplatin are administered to the patient about every 3 weeks (Q3W) for 4 cycles; (b) performing a radical cystectomy; and (c) administering to the patient an adjuvant therapy comprising about 1500 mg of durvalumab about every 4 weeks (Q4W) for up to 8 cycles.

In another aspect, the disclosure provides a combination therapy for use in a method of treating a patient with bladder cancer, the method comprising (a) administering to the patient a combination therapy comprising an anti-PD-L1 antibody or antigen binding fragment thereof and one or more platinum-based chemotherapies; and (b) performing a radical cystectomy

In another aspect, the disclosure provides a combination therapy for use in a method of treating a patient with bladder cancer, the method comprising: (a) administering about 1500 mg of durvalumab and one or more platinum-based chemotherapies, wherein the durvalumab and one or more platinum-based chemotherapies are administered to the patient about every 3 weeks (Q3W) for about 4 cycles; (b) performing a radical cystectomy; and (c) administering an adjuvant therapy comprising about 1500 mg of durvalumab, wherein the adjuvant therapy is administered to the patient about every 4 weeks (Q4W) for up to about 8 cycles.

In another aspect, the disclosure provides a combination therapy for use in treating a patient with bladder cancer, the method comprising administering about 1500 mg of durvalumab, gemcitabine at a dose of about 1000 mg/m², and cisplatin; wherein the cisplatin is administered to the patient at a dose of about 70 mg/m² if the patient has a creatinine clearance of ≥60 mL/min or at a dose of about 35 mg/m² if the patient has a creatinine clearance of ≥40 mL/min to <60 mL/min; and wherein the durvalumab, gemcitabine, and cisplatin are administered to the patient about every 3 weeks (Q3W) for 4 cycles.

In another aspect, the disclosure provides use of a combination therapy comprising an anti-PD-L1 antibody or antigen binding fragment thereof and one or more platinum-based chemotherapies for the manufacture of a medicament for treating bladder cancer in a patient before the patient receives a radical cystectomy.

In another aspect, the disclosure provides use of a combination therapy comprising about 1500 mg durvalumab and one or more platinum-based chemotherapies for the manufacture of a medicament for treating bladder cancer in a patient in need thereof before the patient receives a radical cystectomy; wherein the medicament is administered to the patient about every 3 weeks (Q3W) for about 4 cycles; and wherein an adjuvant therapy comprising about 1500 mg of durvalumab is administered to the patient about every 4 weeks (Q4W) for about 8 cycles following the radical cystectomy.

In another aspect, the disclosure provides use of a combination therapy comprising about 1500 mg durvalumab, gemcitabine, and cisplatin for the manufacture of a medicament for treating bladder cancer in a patient in need thereof before the patient receives a radical cystectomy; wherein the medicament is administered to the patient about every 3 weeks (Q3W) for 4 cycles; wherein gemcitabine is administered to the patient at a dose of about 1000 mg/m² on days 1 and 8 of each cycle; wherein cisplatin is administered to the patient at a dose of about 70 mg/m² on day 1 of each cycle if the patient has a creatinine clearance of about ≥60 mL/min or at a dose of about 35 mg/m² on days 1 and 8 of each cycle if the patient has a creatinine clearance of about ≥40 mL/min to <60 mL/min; and wherein an adjuvant therapy comprising about 1500 mg of durvalumab is administered to the patient about every 4 weeks (Q4W) for up to 8 cycles following the radical cystectomy.

These and other features and advantages of the present disclosure will be more fully understood from the following detailed description taken together with the accompanying claims. It is noted that the scope of the claims is defined by the recitations therein and not by the specific discussion of features and advantages set forth in the present description.

### BRIEF DESCRIPTION OF THE DRAWINGS

The accompanying drawings are included to provide a further understanding of the methods and compositions of the disclosure. The drawings illustrate one or more embodiments of the disclosure, and together with the description serve to explain the principles and operation of the disclosure.

FIG. 1 shows the study design for the phase III, randomized, double-blind, open-label, multi-center global study according to the disclosure. Patients with CrCl≥60 mL/min received cisplatin 70 mg/m² and gemcitabine 1000 mg/m² on Day 1, then gemcitabine 1000 mg/m² on Day 8 every 21 days for 4 cycles; patients with CrCl received≥40-<60 mL/min=received cisplatin 35 mg/m² and gemcitabine 1000 mg/m² on Day 1, then gemcitabine 1000 mg/m² on Day 8 every 21 days for 4 cycles. BCG=Bacillus Calmette Guérin; CrCl=creatinine clearance; ECOF PS=Eastern Cooperative Oncology Group performance score; EFS=event-free survival; IV=intravenous; MIBC=muscle-invasive bladder cancer; OS=overall survival; pCR=pathological complete response; Q3W=every 3 weeks; Q4W=every 4 weeks.

FIG. 2 depicts the neoadjuvant dosing schedule of each cycle represented over the number of weeks commenced in the trial. The vertical arrows represent Day 1 of dosing for each cycle.

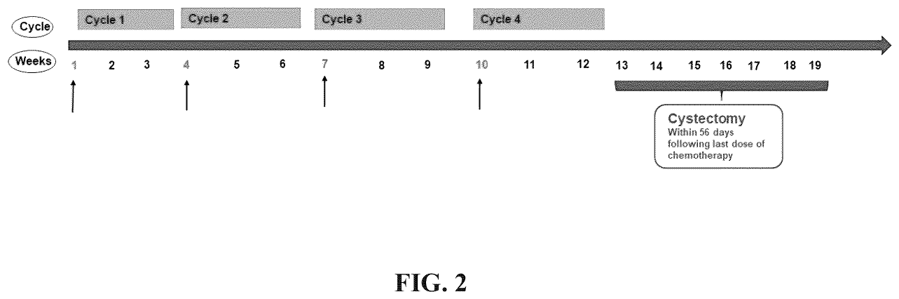

FIG. 3 represents the baseline characteristics of all randomized patients. Baseline characteristics were balanced between study arms. Characteristics included World Health Organization/Eastern Cooperative Oncology Group (WHO/ECOG) performance status, renal function, tumor stage, and histology. CrCl=creatinine clearance; ECOG PS=Eastern Cooperative Oncology Group performance score.

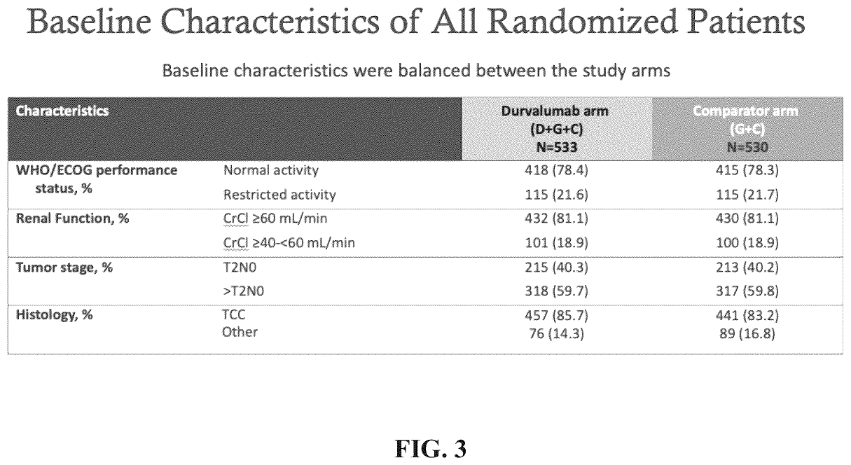

FIG. 4 represents patient disposition and treatment as it pertains to the study phases: neoadjuvant phase, surgery, and adjuvant phase. Data are presented as number (n) and percentage (%). G+C=gemcitabine+cisplatin; D+G+C=durvalumab+gemcitabine+cisplatin

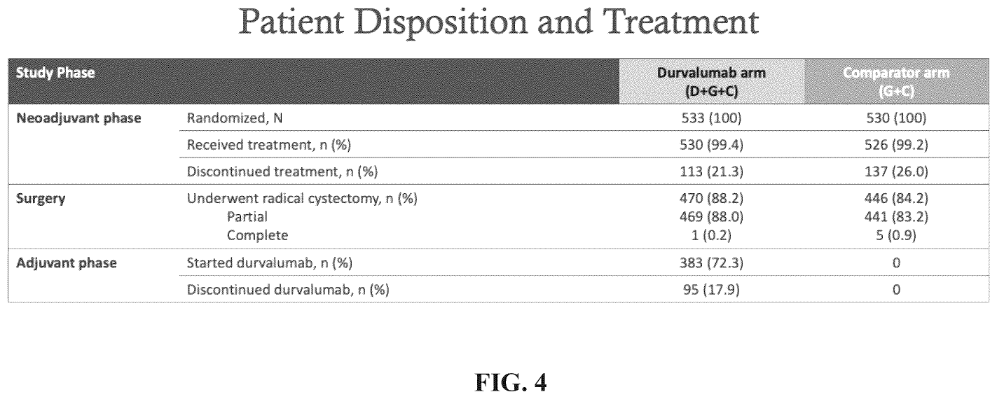

FIG. 5 represents the event-free survival (EFS) using RECIST v1.1 (BICR), depicted by the probability of EFS over time since randomization in months. In accordance with FDA guidance on clinical trial endpoints for the approval of cancer drugs and biologics (FDA 2018), EFS was defined as the time from randomization to the first of any of the following events: (1) the first recurrence of disease post-radical cystectomy, or (2) death due to any cause, or (3) time of first documented progression in patients who were precluded from radical cystectomy, or (4) time of expected surgery in patients who refuse to undergo a radical surgery, or failure to undergo a radical cystectomy in participants with residual disease. BICR=blinded independent central review; CI=confidence interval; DCO=data cut off; EFS=event-free survival; HR=hazard ratio; NR=not reached; RC=radical cystectomy.

FIG. 6 represents pathologic complete response (pCR) between the durvalumab arm and the gemcitabine+cisplatin arm. Results are reported as patients with pCR in each group and the odds ratio. G+C=gemcitabine+cisplatin; D+G+C=durvalumab+gemcitabine+cisplatin.

FIG. 7 represents the interim analysis of overall survival depicted by the probability of survival over time since randomization in months. DCO=Data Cut Off. CI=confidence interval; DCO=data cut off; HR=hazard ratio; NR=not reached; OS=overall survival.

FIG. 8 represents metastasis-free survival in the intent to treat population in both the durvalumab and comparator arms. The probability of metastasis-free survival at 24-months is indicated by the vertical dashed line. Tick marks indicate patients with censored data. CI=confidence interval; D=durvalumab; HR=hazard ratio; ITT=intent-to-treat population; MFS=metastasis-free survival; NAC=neoadjuvant chemotherapy; NR=not reached.

FIG. 9 represents disease-free survival in the intent to treat population in both the durvalumab and comparator arms. The probability of disease-free survival at 24-months is indicated by the vertical dashed line. Tick marks indicate patients with censored data. CI=confidence interval; D=durvalumab; DSS=disease-specific survival; HR=hazard ratio; ITT=intent-to-treat population; NAC=neoadjuvant chemotherapy; NR=not reached.

FIG. 10 represents immune-mediated adverse events (imAE) that were greater than 1% in patients in durvalumab arm in the overall study period. The top number on each bar represents the percentage of patients with any grade imAE and the bottom number on each bar is the grade 3 or 4 imAE in the durvalumab arm.

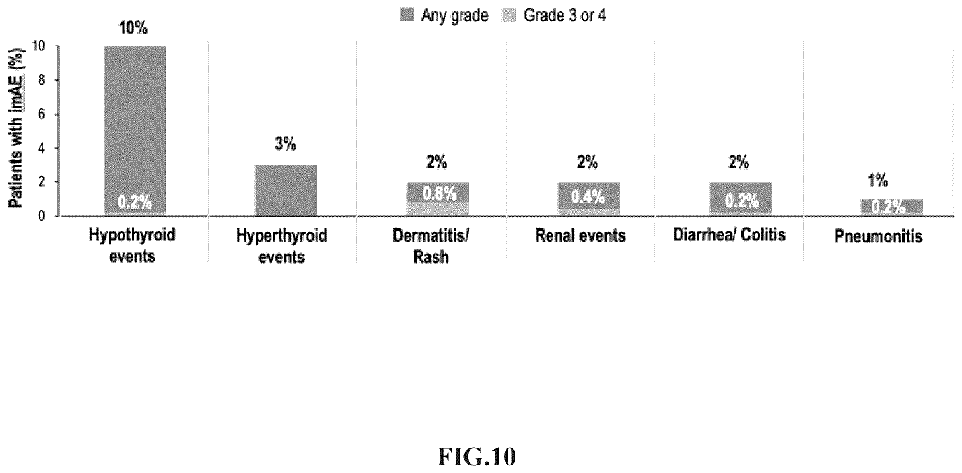

### DETAILED DESCRIPTION OF THE DISCLOSURE

Unless defined otherwise, all technical and scientific terms used herein have the meaning commonly understood by a person skilled in the art to which this disclosure belongs. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

As used herein, the terms “comprise” and “include” and variations thereof (e.g., “comprises,” “comprising,” “includes,” and “including”) will be understood to indicate the inclusion of a stated component, feature, element, or step or group of components, features, elements or steps but not the exclusion of any other component, feature, element, or step or group of components, features, elements, or steps. Any of the terms “comprising,” “consisting essentially of,” and “consisting of” may be replaced with either of the other two terms, while retaining their ordinary meanings.

As used herein, the singular forms “a,” “an,” and “the” also include plural referents unless the context clearly indicates otherwise.

Percentages disclosed herein can vary in amount by +10, 20, or 30% from values disclosed and remain within the scope of the contemplated disclosure.

Unless otherwise indicated or otherwise evident from the context and understanding of one of ordinary skill in the art, values herein that are expressed as ranges can assume any specific value or sub-range within the stated ranges in different embodiments of the disclosure, to the tenth of the unit of the lower limit of the range, unless the context clearly dictates otherwise.

As used herein, ranges and amounts can be expressed as “about” a particular value or range. The term “about” also includes the exact amount. For example, “about 5%” means “about 5%” and also “5%.” The term “about” can also refer to ±10% of a given value or range of values. Therefore, about 5% also means 4.5%-5.5%, for example. Additionally, “about” or “comprising essentially of” can mean a range of up to ±10%. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition. Unless otherwise clear from context, all numerical values provided herein are modified by the term “about.”

As described herein, any concentration range, percentage range, ratio range, or integer range is to be understood to include the value of any integer within the recited range and, when appropriate, fractions thereof (such as one tenth and one hundredth of an integer), unless otherwise indicated.

Units, prefixes, and symbols are denoted in their Système International de Unites (SI) accepted form. Numeric ranges are inclusive of the numbers defining the range. Unless otherwise indicated, nucleotide sequences are written left to right in 5′ to 3′ orientation. Amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

As used herein, the terms “or” and “and/or” can describe multiple components in combination or exclusive of one another. For example, “x, y, and/or z” can refer to “x” alone, “y” alone, “z” alone, “x, y, and z,” “(x and y) or z,” “x or (y and z),” or “x or y or z.”

This disclosure demonstrates that perioperative durvalumab plus neoadjuvant chemotherapy followed by radical cystectomy improves EFS, pCR, 12-month event free survival (EFS12), 24-month event free survival (EFS24), disease-free survival (DFS), 24-month OS, and overall survival (OS).

As utilized in accordance with the present disclosure, unless otherwise indicated, all technical and scientific terms shall be understood to have the same meaning as commonly understood by one of ordinary skill in the art. Unless otherwise required by context, singular terms shall include pluralities, and plural terms shall include the singular.

This disclosure provides for methods of treating a patient having bladder cancer that result in a 12-month event-free survival (EFS12) in at least about 76% of patients. This disclosure provides for methods of treating a patient having bladder cancer that result in a 24-month event-free survival (EFS24) in at least about 67% of patients. This disclosure provides for methods of treating a patient having bladder cancer that produce a pathological complete response (pCR) in at least about 37% of patients. This disclosure provides for methods of treating a patient having bladder cancer that reduce the number of events resulting in an overall survival (OS) in more than 25% of patients.

In an aspect, this disclosure provides a method of treating bladder cancer in a patient in need thereof, the method comprising (a) administering to the patient an anti-PD-L1 antibody or antigen binding fragment thereof and one or more platinum-based chemotherapies; and (b) performing a radical cystectomy.

In one embodiment, this disclosure provides a method of treating bladder cancer, wherein the anti-PD-L1 antibody or antigen binding fragment thereof and one or more platinum-based chemotherapies are administered about every 2 to 4 weeks (Q2W-Q4W) for about 3 to 6 cycles.

In one aspect, this disclosure provides a method of treating bladder cancer, wherein the anti-PD-L1 antibody or antigen binding fragment thereof and one or more platinum-based chemotherapies are administered about every 3 weeks (Q3W) for about 4 cycles.

In one aspect, this disclosure provides a method of treating bladder cancer, wherein the anti-PD-L1 antibody or antigen binding fragment thereof is durvalumab.

In one aspect, this disclosure provides a method of treating bladder cancer, wherein the anti-PD-L1 antibody or antigen binding fragment thereof comprises about 500-2000 mg of durvalumab.

In one aspect, this disclosure provides a method of treating bladder cancer, wherein the anti-PD-L1 antibody or antigen binding fragment thereof comprises about 1500 mg of durvalumab.

In one aspect, this disclosure provides a method of treating bladder cancer, wherein the anti-PD-L1 antibody or antigen binding fragment thereof comprises about 20 mg/kg of durvalumab.

In one aspect, this disclosure provides a method of treating bladder cancer, further comprising administering to the patient an adjuvant therapy comprising an anti-PD1-L1 antibody or antigen binding fragment thereof following the radical cystectomy.

In one aspect, this disclosure provides a method of treating bladder cancer, wherein the adjuvant therapy comprises about 500-2000 mg of durvalumab.

In one aspect, this disclosure provides a method of treating bladder cancer wherein the adjuvant therapy comprises about 1500 mg of durvalumab.

In one aspect, this disclosure provides a method of treating bladder cancer, wherein the adjuvant therapy comprises about 1500 mg of durvalumab administered to the patient about every 2 to 4 weeks (Q2W-Q4W) for up to about 12 cycles.

In one aspect, this disclosure provides a method of treating bladder cancer, wherein the adjuvant therapy comprises about 20 mg/kg of durvalumab.

In one aspect, this disclosure provides a method of treating bladder cancer, wherein the adjuvant therapy is administered to the patient about every 4 weeks (Q4W) for up to about 8 cycles.

In one aspect, this disclosure provides a method of treating bladder cancer, the method comprising (a) administering to the patient about 1500 mg of durvalumab and one or more platinum-based chemotherapies about every 3 weeks (Q3W) for about 4 cycles; (b) performing a radical cystectomy; and (c) administering to the patient an adjuvant therapy comprising about 1500 mg of durvalumab about every 4 weeks (Q4W) for about 8 cycles.

In one aspect, this disclosure provides a method of treating bladder cancer, wherein the one or more platinum-based chemotherapies are afatinib, bevacizumab, carboplatin, cetuximab, cisplatin, doxorubicin, erlotinib, gemcitabine, methotrexate, nedaplatin, oxaliplatin, paclitaxel, pemetrexed, emurafenib, and/or vinblastine.

In one aspect, this disclosure provides a method of treating bladder cancer, wherein the one or more platinum-based chemotherapies are carboplatin and gemcitabine.

In one aspect, this disclosure provides a method of treating bladder cancer, wherein the one or more platinum-based chemotherapies are (a) cisplatin administered to the patient at a dose of about 10-100 mg/m² Q3W; and (b) gemcitabine administered to the patient at a dose of about 500-1500 mg/m² Q3W.

In one aspect, this disclosure provides a method of treating bladder cancer, wherein if the patient has a creatinine clearance of about ≥60 mL/min, the one or more platinum-based chemotherapies are (a) cisplatin administered to the patient at a dose of about 70 mg/m² on day 1 of each cycle; and (b) gemcitabine administered to the patient at a dose of about 1000 mg/m² on days 1 and 8 of each cycle.

In one aspect, this disclosure provides a method of treating bladder cancer, wherein, if the patient has a creatinine clearance of about ≥40 mL/min to <60 mL/min, the one or more platinum-based chemotherapies are (a) cisplatin administered to the patient at a dose of about 35 mg/m² on days 1 and 8 of each cycle; and (b) gemcitabine administered to the patient at a dose of about 1000 mg/m² on days 1 and 8 of each cycle.

In one aspect, this disclosure provides a method of treating bladder cancer in a patient in need thereof, the method comprising: (a) administering to the patient: (i) about 1500 mg of durvalumab; (ii) gemcitabine at a dose of about 1000 mg/m² on days 1 and 8 of each cycle; (iii) cisplatin at a dose of about 70 mg/m² on day 1 of each cycle if the patient has a creatinine clearance of about ≥60 mL/min or at a dose of about 35 mg/m² on days 1 and 8 of each cycle if the patient has a creatinine clearance of about ≥40 mL/min to <60 mL/min; wherein the durvalumab, gemcitabine, and cisplatin are administered to the patient about every 3 weeks (Q3W) for 4 cycles; (b) performing a radical cystectomy; and (c) administering to the patient an adjuvant therapy comprising about 1500 mg of durvalumab about every 4 weeks (Q4W) for up to 8 cycles.

In one aspect, this disclosure provides a method of treating bladder cancer, wherein the anti-PD-L1 antibody or antigen binding fragment thereof is administered to the patient prior to administration of the one or more platinum-based chemotherapies.

In one aspect, this disclosure provides a method of treating bladder cancer, wherein the anti-PD-L1 antibody or antigen binding fragment thereof is administered to the patient at the same time as administration of the one or more platinum-based chemotherapies.

In one aspect, this disclosure provides a method of treating bladder cancer, wherein the radical cystectomy is performed about 14 days to about 70 days following administration of 4 cycles of the anti-PD-L1 antibody or antigen binding fragment thereof and one or more platinum-based chemotherapies.

In one aspect, this disclosure provides a method of treating bladder cancer, wherein the radical cystectomy is preformed about 56 days following administration of 4 cycles of the anti-PD-L1 antibody or antigen binding fragment thereof and one or more platinum-based chemotherapies.

In one aspect, this disclosure provides a method of treating bladder cancer, wherein the adjuvant therapy comprises about 1500 mg of durvalumab administered to the patient every four weeks (Q4W) for up to about 32 weeks.

In one aspect, this disclosure provides a method of treating bladder cancer, wherein the adjuvant therapy is administered to the patient about 28 days to about 150 days following the radical cystectomy.

In one aspect, this disclosure provides a method of treating bladder cancer, wherein the adjuvant therapy is administered to the patient about 42 days to about 120 days following the radical cystectomy.

In one aspect, this disclosure provides a method of treating bladder cancer, wherein the bladder cancer is muscle-invasive bladder cancer (MIBC) or transitional cell carcinoma (TCC) of the urothelium.

In one aspect, this disclosure provides a method of treating bladder cancer, wherein the MIBC is resectable MIBC.

In one aspect, this disclosure provides a method of treating bladder cancer, wherein the patient has not received prior systemic chemotherapy or immunotherapy for treatment of MIBC.

In one aspect, this disclosure provides a combination therapy for use in a method of treating a patient with bladder cancer, the method comprising (a) administering to the patient a combination therapy comprising an anti-PD-L1 antibody or antigen binding fragment thereof and one or more platinum-based chemotherapies; and (b) performing a radical cystectomy.

In one aspect, this disclosure provides a combination therapy for the use in a method of treating a patient with bladder cancer, wherein the anti-PD-L1 antibody or antigen binding fragment thereof and one or more platinum-based chemotherapies are administered about every 2 to 4 weeks (Q2W-Q4W) for about 3 to 6 cycles.

In one aspect, this disclosure provides a combination therapy for the use in a method of treating a patient with bladder cancer wherein the anti-PD-L1 antibody or antigen binding fragment thereof and one or more platinum-based chemotherapies are administered about every 3 weeks (Q3W) for about 4 cycles.

In one aspect, this disclosure provides a combination therapy for the use in a method of treating a patient with bladder cancer, wherein the anti-PD-L1 antibody or antigen binding fragment thereof is durvalumab.

In one aspect, this disclosure provides a combination therapy for the use in a method of treating a patient with bladder cancer, wherein the anti-PD-L1 antibody or antigen binding fragment comprises about 500-2000 mg of durvalumab.

In one aspect, this disclosure provides a combination therapy for the use in a method of treating a patient with bladder cancer, wherein the anti-PD-L1 antibody or antigen binding fragment comprises about 1500 mg of durvalumab.

In one aspect, this disclosure provides a combination therapy for the use in a method of treating a patient with bladder cancer, wherein the anti-PD-L1 antibody or antigen binding fragment thereof comprises about 20 mg/kg of durvalumab.

In one aspect, this disclosure provides a combination therapy for the use in a method of treating a patient with bladder cancer, further comprising administering an adjuvant therapy comprising an anti-PD1-L1 antibody or antigen binding fragment thereof.

In one aspect, this disclosure provides a combination therapy for the use in a method of treating a patient with bladder cancer, wherein the adjuvant therapy comprises about 500-2000 mg of durvalumab.

In one aspect, this disclosure provides a combination therapy for the use in a method of treating a patient with bladder cancer, wherein the adjuvant therapy comprises about 1500 mg of durvalumab.

In one aspect, this disclosure provides a combination therapy for the use in a method of treating a patient with bladder cancer, wherein the adjuvant therapy comprises about 1500 mg of durvalumab administered to the patient every 2 to 4 weeks (Q2W-A4W) for up to about 12 cycles.

In one aspect, this disclosure provides a combination therapy for the use in a method of treating a patient with bladder cancer, wherein the adjuvant therapy is administered to the patient about every 4 weeks (Q4W) for up to about 8 cycles.

In one aspect, this disclosure provides a combination therapy for the use in a method of treating a patient with bladder cancer, the method comprising: (a) administering about 1500 mg of durvalumab and one or more platinum-based chemotherapies, wherein the durvalumab and one or more platinum-based chemotherapies are administered to the patient about every 3 weeks (Q3W) for about 4 cycles; (b) performing a radical cystectomy; and (c) administering an adjuvant therapy comprising about 1500 mg of durvalumab, wherein the adjuvant therapy is administered to the patient about every 4 weeks (Q4W) for up to about 8 cycles.

In one aspect, this disclosure provides a combination therapy for the use in a method of treating a patient with bladder cancer, wherein the one or more platinum-based chemotherapies are afatinib, bevacizumab, carboplatin, cetuximab, cisplatin, doxorubicin, emurafenib, erlotinib, gemcitabine, methotrexate, nedaplatin, oxaliplatin, paclitaxel, pemetrexed, and/or vinblastine.

In one aspect, this disclosure provides a combination therapy for the use in a method of treating a patient with bladder cancer, wherein the one or more platinum-based chemotherapies are cisplatin and gemcitabine.

In one aspect, this disclosure provides a combination therapy for the use in a method of treating a patient with bladder cancer, wherein the one or more platinum-based chemotherapies are (a) cisplatin administered to the patient at a dose of about 10-100 mg/m² Q3W and (b) gemcitabine administered to the patient at a dose of about 500-1500 mg/m² Q3W.

In one aspect, this disclosure provides a combination therapy for the use in a method of treating a patient with bladder cancer, wherein, if the patient has a creatinine clearance of about ≥ 60 mL/min, the one or more platinum-based chemotherapies are (a) cisplatin administered to the patient at a dose of about 70 mg/m² on day 1 of each cycle; and (b) gemcitabine administered to the patient at a dose of about 1000 mg/m² on days 1 and 8 of each cycle.

In one aspect, this disclosure provides a combination therapy for the use in a method of treating a patient with bladder cancer, wherein, if the patient has a creatinine clearance of about ≥ 40 mL/min to <60 mL/min, the one or more platinum-based chemotherapies are (a) cisplatin administered to the patient at a dose of about 35 mg/m² on days 1 and 8 of each cycle; and (b) gemcitabine administered to the patient at a dose of about 1000 mg/m² on days 1 and 8 of each cycle.

In one aspect, this disclosure provides a combination therapy for the use in a method of treating a patient with bladder cancer, the method comprising administering about 1500 mg of durvalumab, gemcitabine at a dose of about 1000 mg/m², and cisplatin; wherein the cisplatin is administered to the patient at a dose of about 70 mg/m² if the patient has a creatinine clearance of ≥60 mL/min or at a dose of about 35 mg/m² if the patient has a creatinine clearance of ≥40 mL/min to <60 mL/min; and wherein the durvalumab, gemcitabine, and cisplatin are administered to the patient about every 3 weeks (Q3W) for 4 cycles.

In one aspect, this disclosure provides a combination therapy for the use in a method of treating a patient with bladder cancer, further comprising administering an adjuvant therapy comprising an anti-PD1-L1 antibody or antigen binding fragment thereof.

In one aspect, this disclosure provides a combination therapy for the use in a method of treating a patient with bladder cancer, wherein the adjuvant therapy comprises about 500-2000 mg of durvalumab.

In one aspect, this disclosure provides a combination therapy for the use in a method of treating a patient with bladder cancer, wherein the adjuvant therapy comprises about 1500 mg of durvalumab.

In one aspect, this disclosure provides a combination therapy for the use in a method of treating a patient with bladder cancer, wherein the adjuvant therapy comprises about 20 mg/kg of durvalumab.

In one aspect, this disclosure provides a combination therapy for the use in a method of treating a patient with bladder cancer, wherein the adjuvant therapy comprises about 1500 mg of durvalumab administered to the patient every 2 to 4 weeks (Q2W-Q4W) for up to about 12 cycles.

In one aspect, this disclosure provides a combination therapy for the use in a method of treating a patient with bladder cancer, wherein the adjuvant therapy is administered to the patient about every 4 weeks (Q4W) for up to about 8 cycles.

In one aspect, this disclosure provides a combination therapy for the use in a method of treating a patient with bladder cancer, wherein the anti-PD-L1 antibody or antigen binding fragment thereof is administered to the patient prior to administration of the one or more platinum-based chemotherapies.

In one aspect, this disclosure provides a combination therapy for the use in a method of treating a patient with bladder cancer, wherein the anti-PD-L1 antibody or antigen binding fragment thereof is administered to the patient at the same time as administration of the one or more platinum-based chemotherapies.

In one aspect, this disclosure provides a combination therapy for the use in a method of treating a patient with bladder cancer, wherein a radical cystectomy is performed about 14 days to about 70 days following administration of 4 cycles of the anti-PD-L1 antibody or antigen binding fragment thereof and one or more platinum-based chemotherapies.

In one aspect, this disclosure provides a combination therapy for the use in a method of treating a patient with bladder cancer, wherein the radical cystectomy is preformed about 56 days following administration of 4 cycles of the anti-PD-L1 antibody or antigen binding fragment thereof and one or more platinum-based chemotherapies.

In one aspect, this disclosure provides a combination therapy for the use in a method of treating a patient with bladder cancer, wherein the adjuvant therapy comprises about 1500 mg of durvalumab administered to the patient every 4 weeks (Q4W) for up to about 8 cycles.

In one aspect, this disclosure provides a combination therapy for the use in a method of treating a patient with bladder cancer, wherein the adjuvant therapy is administered to the patient about 28 days to about 150 days following a radical cystectomy.

In one aspect, this disclosure provides a combination therapy for the use in a method of treating a patient with bladder cancer, wherein the adjuvant therapy is administered to the patient about 42 days to about 120 days following the radical cystectomy.

In one aspect, this disclosure provides a combination therapy for the use in a method of treating a patient with bladder cancer, wherein the bladder cancer is muscle-invasive bladder cancer (MIBC) or transitional cell carcinoma (TCC) of the urothelium.

In one aspect, this disclosure provides a combination therapy for the use in a method of treating a patient with bladder cancer, wherein the MIBC is resectable MIBC.

In one aspect, this disclosure provides a use of a combination therapy comprising an anti-PD-L1 antibody or antigen binding fragment thereof and one or more platinum-based chemotherapies for the manufacture of a medicament for treating bladder cancer in a patient before the patient receives a radical cystectomy.

In one aspect, this disclosure provides the use of a combination therapy, wherein the medicament is administered to the patient about every 2 to 4 weeks (Q2W-Q4W) for about 3 to 6 cycles.

In one aspect, this disclosure provides the use of a combination therapy, wherein the medicament is administered to the patient about every 3 weeks (Q3W) for about 4 cycles.

In one aspect, this disclosure provides the use of a combination therapy, wherein the anti-PD-L1 antibody or antigen binding fragment thereof is durvalumab.

In one aspect, this disclosure provides the use of a combination therapy, wherein the medicament comprises about 500-2000 mg of durvalumab.

In one aspect, this disclosure provides the use of a combination therapy, wherein the medicament comprises about 1500 mg of durvalumab.

In one aspect, this disclosure provides the use of a combination therapy, wherein the medicament comprises about 20 mg/kg of durvalumab.

In one aspect, this disclosure provides the use of a combination therapy, further comprising an adjuvant therapy comprising an anti-PD1-L1 antibody or antigen binding fragment thereof, wherein the adjuvant therapy is administered to the patient after the patient receives a radical cystectomy.

In one aspect, this disclosure provides the use of a combination therapy, wherein the adjuvant therapy comprises about 500-2000 mg of durvalumab.

In one aspect, this disclosure provides the use of a combination therapy, wherein the adjuvant therapy comprises about 1500 mg of durvalumab.

In one aspect, this disclosure provides the use of a combination therapy, wherein the adjuvant therapy comprises about 1500 mg of durvalumab administered to the patient about every 2 to 4 weeks (Q2W-Q4W) for up to about 12 cycles.

In one aspect, this disclosure provides the use of a combination therapy, wherein the adjuvant therapy is administered to the patient about every 4 weeks (Q4W) for up to about 8 cycles.

In one aspect, this disclosure provides the use of a combination therapy, wherein the adjuvant therapy comprises about 20 mg/kg of durvalumab administered to the patient about every 2 to 4 weeks (Q2W-Q4W) for up to about 12 cycles.

In one aspect, this disclosure provides the use of a combination therapy, wherein the adjuvant therapy comprises about 20 mg/kg of durvalumab administered to the patient about every 3 weeks (Q2W-Q4W) for up to about 8 cycles.

In one aspect, this disclosure provides the use of a combination therapy comprising about 1500 mg durvalumab and one or more platinum-based chemotherapies for the manufacture of a medicament for treating bladder cancer in a patient in need thereof before the patient receives a radical cystectomy; wherein the medicament is administered to the patient about every 3 weeks (Q3W) for about 4 cycles; and wherein an adjuvant therapy comprising about 1500 mg of durvalumab is administered to the patient about every 4 weeks (Q4W) for about 8 cycles following the radical cystectomy.

In one aspect, this disclosure provides the use of a combination therapy, wherein the one or more platinum-based chemotherapies is afatinib, bevacizumab, carboplatin, cetuximab, cisplatin, doxorubicin, emurafenib, erlotinib, gemcitabine, methotrexate, nedaplatin, oxaliplatin, paclitaxel, pemetrexed, and/or vinblastine.

In one aspect, this disclosure provides the use of a combination therapy, wherein the one or more platinum-based chemotherapies are carboplatin and gemcitabine.

In one aspect, this disclosure provides the use of a combination therapy, wherein the one or more the platinum-based chemotherapies are (a) cisplatin administered to the patient at a dose of about 10-100 mg/m² Q3W; and (b) gemcitabine administered to the patient at a dose of about 500-1500 mg/m² Q3W.

In one aspect, this disclosure provides use of a combination therapy, wherein, if the patient has a creatinine clearance of about ≥60 mL/min, the one or more platinum-based chemotherapies are (a) cisplatin administered to the patient at a dose of about 70 mg/m² on day 1 of each cycle; and (b) gemcitabine administered to the patient at a dose of about 1000 mg/m² on days 1 and 8 of each cycle.

In one aspect, this disclosure provides the use of a combination therapy, wherein if the patient has a creatinine clearance of about ≥40 mL/min to <60 mL/min, the one or more platinum-based chemotherapies are (a) cisplatin administered to the patient at a dose of about 35 mg/m² on days 1 and 8 of each cycle; and (b) gemcitabine administered to the patient at a dose of about 1000 mg/m² on days 1 and 8 of each cycle.

In one aspect, this disclosure provides the use of a combination comprising about 1500 mg durvalumab, gemcitabine, and cisplatin for the manufacture of a medicament for treating bladder cancer in a patient in need thereof before the patient receives a radical cystectomy; wherein the medicament is administered to the patient about every 3 weeks (Q3W) for 4 cycles; wherein gemcitabine is administered to the patient at a dose of about 1000 mg/m² on days 1 and 8 of each cycle; wherein cisplatin is administered to the patient at a dose of about 70 mg/m² on day 1 of each cycle if the patient has a creatinine clearance of about ≥60 mL/min or at a dose of about 35 mg/m² on days 1 and 8 of each cycle if the patient has a creatinine clearance of about ≥40 mL/min to <60 mL/min; and wherein an adjuvant therapy comprising about 1500 mg of durvalumab is administered to the patient about every 4 weeks (Q4W) for up to 8 cycles following the radical cystectomy.

In one aspect, this disclosure provides the use of a combination therapy, wherein the anti-PD-L1 antibody or antigen binding fragment thereof is administered to the patient prior to administration of the one or more platinum-based chemotherapies.

In one aspect, this disclosure provides the use of a combination therapy, wherein the anti-PD-L1 antibody or antigen binding fragment thereof is administered to the patient at the same time as administration of the one or more platinum-based chemotherapies.

In one aspect, this disclosure provides the use of a combination therapy, wherein the patient receives a radical cystectomy about 14 days to about 70 days following administration of 4 cycles of the anti-PD-L1 antibody or antigen binding fragment thereof and one or more platinum-based chemotherapies.

In one aspect, this disclosure provides the use of a combination therapy, wherein the patient receives a radical cystectomy about 56 days following administration of 4 cycles of the anti-PD-L1 antibody or antigen binding fragment thereof and one or more platinum-based chemotherapies.

In one aspect, this disclosure provides the use of a combination therapy, wherein the adjuvant therapy comprises about 1500 mg of durvalumab administered to the patient every 4 weeks (Q4W) for up to about 8 cycles.

In one aspect, this disclosure provides the use of a combination therapy, wherein the adjuvant therapy is administered to the patient about 28 days to about 150 days after the patient receives a radical cystectomy.

In one aspect, this disclosure provides the use of a combination therapy, wherein the adjuvant therapy is administered to the patient about 42 days to about 120 days after the patient receives a radical cystectomy.

In one aspect, this disclosure provides the use of a combination therapy, wherein the bladder cancer is muscle-invasive bladder cancer (MIBC) or transitional cell carcinoma (TCC) of the urothelium.

In one aspect, this disclosure provides the use of a combination therapy, wherein the MIBC is resectable MIBC.

In one aspect, this disclosure provides the use of a combination therapy, wherein the patient has not received prior systemic chemotherapy or immunotherapy for treatment of MIBC.

In one aspect, this disclosure provides the use of a combination therapy comprising about 20 mg/kg of durvalumab and one or more platinum-based chemotherapies for the manufacture of a medicament for treating muscle invasive bladder cancer (MIBC) in a patient in need thereof; wherein the medicament is administered to the patient about every 3 to 4 weeks (Q3W-Q4W) for about 4 cycles before the patient receives a radical cystectomy; and wherein an adjuvant therapy comprising about 1500 mg of durvalumab is administered to the patient about every four weeks (Q4W) for at least about 8 cycles following the radical cystectomy.

In one aspect, this disclosure provides the use of a combination therapy, wherein the patient has a creatinine clearance rate of ≥40 mL/min to <60 mL/min and the one or more platinum-based chemotherapies comprise cisplatin at a dose of 35 mg/m² and gemcitabine at a dose of 1000 mg/m² wherein the cisplatin and gemcitabine are administered to the patient once every 3 weeks (Q3W) if the patient has a creatinine clearance rate of ≥40 mL/min to <60 mL/min; and wherein cisplatin is administered to the patient on day 1 of each cycle, and gemcitabine is administered to the patient on days 1 and 8 of each cycle.

In one aspect, this disclosure provides the use of a combination therapy, wherein the patient has a creatinine clearance rate of ≥60 mL/min and the one or more platinum-based chemotherapies comprise cisplatin at a dose of 70 mg/m² and gemcitabine at a dose of 1000 mg/m²; wherein the cisplatin and gemcitabine are administered to the patient once every 3 weeks (Q3W); and wherein cisplatin is administered to the patient on day 1 of each cycle, and gemcitabine is administered to the patient on days 1 and 8 of each cycle.

As used herein, the terms “treat,” “treatment,” or “treating” when used in the context of treating cancer refer to reducing disease pathology, reducing or eliminating disease symptoms, promoting increased survival rates, and/or reducing discomfort. For example, treating can refer to the ability of a therapy when administered to a subject, to reduce disease symptoms, signs, or causes. Treating also refers to mitigating or decreasing at least one clinical symptom and/or inhibition or delay in the progression of the condition and/or prevention or delay of the onset of a disease or illness.

As used herein, the terms “subject,” “individual,” or “patient,” refer to any subject, particularly a mammalian subject, for whom diagnosis, prognosis, or therapy is desired. Mammalian subjects include, for example, humans, non-human primates, dogs, cats, guinea pigs, rabbits, rats, mice, horses, cattle, bears, and so on. The term “patient” may refer to a human. In an aspect, this disclosure provides methods of treating a patient identified as having. The term “bladder cancer” as used herein includes transitional cell and mixed transitional/non-transitional cell histologies such as adenocarcinoma and squamous cell); variant transitional such as micropapillary, plasmacytoid, sarcomatoid, nested variant, lymphoepitheliod, nested variant) histologies. In a preferred embodiment the bladder cancer is muscle invasive bladder cancer (MIBC). In some embodiments the MIBC is resectable. In some embodiments, the patient with MIBC is previously untreated. In some embodiments, the patient with MIBC is previously untreated with chemotherapy and/or immunotherapy. In some embodiments, the patient has muscle-invasive transitional cell carcinoma (also known as UC) of the bladder or transitional cell carcinoma (TCC) of the urothelium. In some embodiments, the patient has resectable transitional cell carcinoma (TCC) of the urothelium. In some embodiments, the patient has transitional cell carcinoma of the urothelium, excluding renal pelvis and ureters. In some embodiments, the patient has histologically or cytologically confirmed MIBC or TCC.

In some embodiments, the patient has MIBC that is histologically or cytologically documented as having tumor stage T2N0 versus greater than T2N0 (including T2N1, T3, and T4a). This reflects prognostic stage II versus stage IIIa (respectively) (according to the American Joint Committee on Cancer [AJCC] Cancer Staging Manual, version 8). In some embodiments, T1-T4 patients with node-positive disease (regional lymph nodes) are classified as IIIa (cN1 disease) and IIIb (N2/N3 disease). In some embodiments, the patient is a T1-T4 patient with node-positive disease. In some embodiments, the regional lymph nodes are positive for MIBC. In some embodiments, the patient is a stage II patient or a stage IIIa patient (T3-T4NO and T1-T4N1).

In some embodiments, the patient has histologically or cytologically confirmed, resectable MIBC (i.e., T2N0/1M0-T4aN0/1M0) and transitional cell carcinoma (TCC; transitional cell and mixed transitional/non-transitional cell histologies) of the urothelium (excluding renal pelvis and ureters) who are candidates for radical cystectomy and have not received prior systemic chemotherapy or immunotherapy for treatment of MIBC.

In some embodiments, the patient has previously untreated resectable MIBC (stage IIA to select [N2] stage IIIB according to the American Joint Committee on Cancer [AJCC] Cancer Staging Manual, version 8).

In some embodiments, PD-L1 status is determined based on a Ventana assay.

In one aspect, a first “neoadjuvant baseline scan” is obtained from each patient for disease staging and for use as a RECIST 1.1 baseline for the post-neoadjuvant/pre-radical cystectomy follow-up scan that is performed upon completion of neoadjuvant chemotherapy prior to surgery to confirm patient is still eligible for a radical cystectomy. In one aspect, single tumor (T)-stage is determined and is used for documentation of baseline disease characteristics and also for registering the patient for randomization/stratification. Clinical staging, specifically for the determination of the clinical tumor stage (CT), is a composite of combined results obtained from a pathological assessment of the tumor (from a TURBT sample, confirming muscle invasion), an examination under anesthesia procedure and results from a CT/MRI image.

A second “Adjuvant Baseline” is obtained at 42 days (±2 weeks) after the radical cystectomy and is performed as close as possible and must be prior to the first date of adjuvant therapy. In the event any radiologically observable tumors are identified, a new selection of Target and/or Non-Target lesions are recorded, and a follow-up scan is performed at least 4 weeks later, as an assessment using RECIST 1.1 criteria and then every 12 weeks, thereafter. On-study adjuvant tumor assessments occur every 12 weeks±7 days after the date of radical cystectomy for the first 24 months, then every 24 weeks±7 days for 36 months, and then every 52 weeks (annually) thereafter until unequivocal progression, the end of study, death, study discontinuation, or Sponsor decision, whichever comes first.

In some embodiments, the patient's tumor PD-L1 status of the MIBC cells is determined prior to treatment. In some embodiments, the MIBC of the patient has a PD-L1 tumor cell expression<1%. In some embodiments, the MIBC of the patient has a PD-L1 tumor cell expression 1-49%. In some embodiments, the MIBC of the patient has a PD-L1 tumor cell expression≥50%. In certain embodiments, a patient's tumor PD-L1 status is determined using Ventana PD-L1 (SP263) immunohistochemistry (IHC) assay. The assay may be used with a formalin fixed paraffin embedded tissue sample.

In some embodiments, the patient has a histologically or cytologically documented muscle-invasive transitional cell carcinoma (TCC) of the bladder. In some embodiments, the patient has transitional cell and mixed transitional/non-transitional cell histologies (adenocarcinoma, squamous cell)/variant transitional (e.g., micropapillary, plasmacytoid, sarcomatoid, nested variant, lymphoepitheliod, nested variant) histologies. In some embodiments, the patient has clinical tumor stage T2-T4aN0/1M0 (AJCC Cancer Staging Manual, 8th Edition) TCC of the bladder. In some embodiments, cN1 disease is identified as the presence of a single lymph node in the true pelvis (i.e., perivesical, obturator, internal and external iliac, and sacral lymph node). In some embodiments, the lymph node is <20 mm in the short axis (small volume metastasis) and resectable.

As used herein, the term “durvalumab” refers to an antibody that selectively binds PD-L1 and blocks the binding of PD-L1 to the PD-1 and CD80 receptors. The durvalumab antibody is disclosed in U.S. Pat. No. 9,493,565 (referred to as “2.14H9OPT”), which is incorporated by reference herein in its entirety. The fragment crystallizable (Fc) domain of durvalumab contains a triple mutation in the constant domain of the IgG1 heavy chain that reduces binding to the complement component C1q and the Fcγ receptors responsible for mediating antibody-dependent cell-mediated cytotoxicity (“ADCC”). In certain embodiments, the triple mutation refers to the IgG1 Fc region comprising a L234F/L235E/P331S triple mutation (EU numbering; see also U.S. Pat. No. 9,493,565). Durvalumab can relieve PD-L1-mediated suppression of human T-cell activation in vitro and inhibits tumor growth in a xenograft model via a T-cell dependent mechanism. The amino acid sequences of durvalumab are as follows:

Durvalumab sequences

MEDI4736 VL
EIVLTQSPGTLSLSPGERATLSCRASQRVSSSYLAWYQQKPGQAPRLLI
YDASSRATGIPDRFSGSGSGTDFTLTISRLEPEDFAVYYCQQYGSLPWT
FGQGTKVEIK (SEQ ID NO: 1)
MEDI4736 VL CDR1
RASQRVSSSYLA (SEQ ID NO: 2)
MEDI4736 VL CDR2
DASSRAT (SEQ ID NO: 3)
MEDI4736 VL CDR3
QQYGSLPWT (SEQ ID NO: 4)
MEDI4736 VH
EVQLVESGGGLVQPGGSLRLSCAASGFTFSRYWMSWVRQAPGKGLEWVA
NIKQDGSEKYYVDSVKGRFTISRDNAKNSLYLQMNSLRAEDTAVYYCAR
EGGWFGELAFDYWGQGTLVTVSS (SEQ ID NO: 5)
MEDI4736 VH CDR1
GFTFSRYWMS (SEQ ID NO: 6)
MEDI4736 VH CDR2
NIKQDGSEKYYVDSVKG (SEQ ID NO: 7)
MEDI4736 VH CDR3
EGGWFGELAFDY (SEQ ID NO: 8)

As used herein, the term “platinum-based chemotherapy” refers to chemotherapy drugs that contain the element platinum. Platinum-based chemotherapy has been used to treat many different types of cancer. In some embodiments, a platinum-based chemotherapy refers to chemotherapy treatment comprising comprises at least one of one or more of afatinib, bevacizumab, carboplatin, cetuximab, cisplatin, doxorubicin, erlotinib, gemcitabine, methotrexate, nedaplatin, oxaliplatin, paclitaxel, pemetrexed, emurafenib, and/or vinblastine. In some embodiments, a platinum-based chemotherapy can also refer to chemotherapy treatment comprising at least one or more of triplatin tetranitrate, phenanthriplatin, picoplatin, and satraplatin. In certain embodiments, a platinum-based chemotherapy further comprises one or more of afatinib, cetuximab, bevacizumab, erlotinib, gemcitabine, paclitaxel, pemetrexed, and vemurafenib. In certain embodiments, a platinum-based chemotherapy comprises carboplatin and paclitaxel. In certain embodiments, a platinum-based chemotherapy comprises cisplatin and gemcitabine. In certain embodiments, a platinum-based chemotherapy comprises carboplatin and gemcitabine. In certain embodiments, a platinum-based chemotherapy comprises carboplatin and pemetrexed. In certain embodiments, a platinum-based chemotherapy comprises cisplatin and pemetrexed. In certain embodiments, a platinum-based chemotherapy comprises a cisplatin dose of about 10-100 mg/m². In some embodiments, a platinum-based chemotherapy comprises a cisplatin dose of about 20-90 mg/m². In some embodiments, a platinum-based chemotherapy comprises a cisplatin dose of about 30-80 mg/m². In some embodiments, a platinum-based chemotherapy comprises a cisplatin dose of about 40-70 mg/m². In some embodiments, a platinum-based chemotherapy comprises a cisplatin dose of about 50-60 mg/m². In some embodiments, a platinum-based chemotherapy comprises a cisplatin dose of about 10 mg/m², 20 mg/m², 30 mg/m², 40 mg/m², 50 mg/m², 60 mg/m², 70 mg/m², 80 mg/m², 90 mg/m², or 100 mg/m². In certain embodiments, a platinum-based chemotherapy comprises a gemcitabine dose of about 500-1500 mg/m². In some embodiments, a platinum-based chemotherapy comprises a gemcitabine dose of about 600-1400 mg/m². In some embodiments, a platinum-based chemotherapy comprises a gemcitabine dose of about 700-1300 mg/m². In some embodiments, a platinum-based chemotherapy comprises a gemcitabine dose of about 800-1200 mg/m². In some embodiments, a platinum-based chemotherapy comprises a gemcitabine dose of about 900-1100 mg/m². In some embodiments, a platinum-based chemotherapy comprises a gemcitabine dose of about 1000 mg/m². In some embodiments, a platinum-based chemotherapy comprises a doublet combination of a cisplatin dose of about 10-100 mg/m² and gemcitabine dose of about 500-1500 mg/m². In some embodiments, a platinum-based chemotherapy comprises a cisplatin dose of about 10-100 mg/m² and gemcitabine dose of about 500-1500 mg/m². In certain embodiments, the doublet combination is dependent on the renal function of the patient, as assessed by creatinine clearance (CrCl) rates.

In some embodiments, a platinum-based chemotherapy doublet combination comprises a cisplatin dose of about 70 mg/m² on day 1 of each 3-week cycle and a gemcitabine dose of about 1000 mg/m² on Days 1 and 8 of each 3-week cycle, if the patient has a creatine clearance rate of >60 mL/min, for a maximum of 4 cycles.

In some embodiments, a platinum-based chemotherapy doublet combination comprises a cisplatin dose of about 35 mg/m² on days 1 and 8 of each 3-week cycle and gemcitabine dose of about 1000 mg/m² on day 1 and 8 of each 3-week cycle if the patient has a creatine clearance rate of about >40 mL/min to about <60 mL/min for a maximum of 4 cycles.

In some embodiments, the combination therapy may be neoadjuvant durvalumab and neoadjuvant chemotherapy. In some embodiments, the combination therapy may be perioperative durvalumab and neoadjuvant chemotherapy. In some embodiments, the combination therapy may comprise neoadjuvant durvalumab, neoadjuvant chemotherapy, and optionally adjuvant durvalumab.

In one embodiment, the combination therapy comprises administering durvalumab about every two weeks to about every four weeks (Q2W to Q4W) to the patient. In one embodiment, the combination therapy comprises administering durvalumab about every three weeks (Q3W) to the patient. In one embodiment, the combination therapy comprises administering about 500 mg to about 2000 mg of durvalumab about every two weeks to about every four weeks (Q2W to Q4W) to the patient. In one embodiment, the combination therapy comprises administering about 1500 mg of durvalumab about every two weeks to about every four weeks (Q2W to Q4W) to the patient. In one embodiment, the combination therapy comprises administering about 500 mg to about 2000 mg of durvalumab about every three weeks (Q3W) to the patient. In one embodiment, the combination therapy comprises administering about 1500 mg of durvalumab about every three weeks (Q3W) to the patient. In one embodiment, the combination therapy comprises administering platinum-based chemotherapy and durvalumab about every two weeks to about every four weeks (Q2W to Q4W) to the patient. In one embodiment, the combination therapy comprises administering platinum-based chemotherapy and durvalumab about every three weeks (Q3W) to the patient.

In some embodiments, the combination therapy comprises administering about 500 mg to about 2000 mg durvalumab once about every two weeks to about every four weeks for up to about 3 to 6 cycles prior to surgery and then about 500 mg to about 2000 mg durvalumab once about every two weeks to about every four weeks for up to about 12 cycles following the bladder surgery. In some embodiments, the combination therapy comprises administering about 1500 mg durvalumab once about every three weeks (Q3W) for up to a maximum of about 4 cycles as neoadjuvant treatment prior to surgery and then about 1500 mg durvalumab once about every four weeks (Q4W) for about 8 cycles following the surgery as an adjuvant therapy. In some embodiments, the surgery performed is a radical cystectomy. In some embodiments the surgery performed is a partial cystectomy. If a patient's weight falls to 30 kg or below (≤30 kg), the patient should receive weight-based dosing equivalent to about 20 mg/kg of durvalumab about Q3W or about Q4W until the weight improves to >30 kg, at which point the patient should start receiving the fixed dosing of durvalumab about 1500 mg about Q3W or about Q4W. During the neoadjuvant treatment phase, patients who have Response Evaluation Criteria in Solid Tumors, version 1.1 (RECIST 1.1)-defined radiological progression may continue to the adjuvant portion of treatment if the progression event did not preclude the patient from having a radical cystectomy, that is the progression is local and/or limited to regional lymph nodes that will be removed at radical cystectomy.

In some embodiments, durvalumab can be provided as a 500-mg vial solution for infusion after dilution. In some embodiments, the solution contains 50 mg/mL durvalumab, 26 mM histidine/histidine-hydrochloride, 275 mM trehalose dihydrate, and 0.02% w/v polysorbate 80; it has a pH of 6.0 and density of 1.054 g/mL. In certain embodiments, a dose of about 1500 mg (for patients>30 kg in WT) can be administered using an IV bag containing 0.9% (w/v) saline or 5% (w/v) dextrose, with a final durvalumab concentration ranging from 1 to 15 mg/mL and delivered through an IV administration set with a 0.2- or 0.22-μm filter. If patient weight falls to ≤30 kg, WT-based dosing at 20 mg/kg can be administered using an IV bag selected such that the final concentration is within 1 to 15 mg/mL. In some embodiments, a standard infusion time is 1 hour (±10 minutes); however, if there are interruptions, the total allowed time must not exceed 8 hours at room temperature.

In an aspect, this disclosure provides methods of treating a patient having bladder cancer, including muscle invasive bladder cancer, comprising (i) administering to the patient about 500-2000 mg of durvalumab and a platinum-based chemotherapy about every two weeks to about every four weeks for about 3 to 6 cycles; (ii) performing a radical cystectomy; and (iii) administering to the patient about 500-2000 mg of durvalumab about every two weeks to about every four weeks for up to 8 cycles.

In an aspect, this disclosure provides methods of treating a patient having bladder cancer, including muscle invasive bladder cancer, comprising (i) administering to the patient about 1500 mg of durvalumab and a platinum-based chemotherapy about every 21 days for about 4 cycles; (ii) performing a radical cystectomy; and (iii) administering to the patient about 1500 mg of durvalumab about every 28 days for up to 8 cycles.

In certain embodiments, after the last dose of the neoadjuvant combination therapy, the radical cystectomy or other surgery to remove all, or a portion, of the bladder and/or lymph nodes should be performed within 56-70 days. In certain embodiments, patients should start adjuvant durvalumab administration as soon as clinically feasible and within about 42-120 days following the radical cystectomy or related surgery.

In some embodiments, the success of the method of treatment is determined by an improvement in pathological complete response (pCR) in patients receiving durvalumab, as compared to patients in the comparator arm treated with neoadjuvant gemcitabine and cisplatin. In some embodiments, the improvement in pCR may be at least about 9 months. In some embodiments the improvement in pCR is about 5% or greater. In some embodiments the improvement in pCR is about 10%.

In some embodiments, the success of the method of treatment is determined by an improvement in event-free survival (EFS) in patients receiving durvalumab, as compared to patients in the comparator arm treated with neoadjuvant gemcitabine and cisplatin. In some embodiments the improvement in EFS is about 20% or greater. In some embodiments the improvement in EFS is about 30% or greater. In some embodiments the improvement in EFS is about 32%.

In some embodiments, the success of the method of treatment is determined by an improvement in disease-free survival as compared to patients treated with neoadjuvant gemcitabine and cisplatin.

In some embodiments, the success of the method of treatment is determined by an improvement in Overall Survival (OS) in patients receiving durvalumab, as compared to patients in the comparator arm treated with neoadjuvant gemcitabine and cisplatin. In some embodiments the improvement in OS is about 10% or greater. In some embodiments the improvement in OS is about 20% or greater. In some embodiments the improvement in OS is about 25%

In some embodiments, the success of the method of treatment is determined by an improvement in metastasis-free survival. In some embodiments, the success of the method of treatment is determined by an improvement in distant metastases risk. In some embodiments the success of the method of treatment is determined by an improvement in disease-specific survival. In some embodiments the success of the method of treatment is determined by reduction in the risk from bladder cancer.

In some embodiments, neoadjuvant gemcitabine and cisplatin can refer to treating a patient having a creatinine clearance of about 60 mL/min or greater with cisplatin 70 mg/m² via IV infusion on Day 1 of each 3-week cycle, for 4 cycles, and gemcitabine 1000 mg/m² via IV infusion on Day 1 and Day 8 of each 3-week cycle, for 4 cycles. In some embodiments, neoadjuvant gemcitabine and cisplatin can refer to treating a patient having a creatinine clearance of about >40 mL/min to <60 mL/min with cisplatin 35 mg/m² via IV infusion on Day 1 and Day 8 of each 3-week cycle, for 4 cycles, and gemcitabine 1000 mg/m² via IV infusion on Day 1 and Day 8 of each 3-week cycle, for 4 cycles.

As used herein “event free survival” (EFS) refers to the time from randomization to the first of any of the following events: (1) the first recurrence of disease post-radical cystectomy, or (2) death due to any cause, or (3) time of first documented progression in patients who were precluded from radical cystectomy, or (4) time of expected surgery in patients who refuse to undergo a radical surgery, or failure to undergo a radical cystectomy in participants with residual disease.

In some embodiments, additional endpoints are examined to further evaluate the antitumor effect of platinum drug therapy with or without durvalumab. In some embodiments, the endpoints include EFS rate at 24 months (EFS24) or EFS rate at 12 months (EFS12). In some embodiments, additional endpoints include metastasis-free survival (MFS), disease specific survival (DSS), proportion of patients who achieve<P2, and proportion of patients alive at 5 years after randomization (OS5).

As used herein, the terms “overall survival” and “OS” refer to the time from the date of randomization until death due to any cause regardless of whether the subject withdraws from randomized therapy or receives another anticancer therapy. Any patient not known to have died at the time of analysis will be censored based on the last recorded date on which the patient was known to be alive. Overall survival can be analyzed in the population using the same methodology as described for EFS. The effect of treatment can be estimated by the HR together with its corresponding CI. Kaplan-Meier plots can be presented by treatment arm. Analyses are performed using Kaplan-Meier estimates of OS to estimate the number of OS patients at 12, 24, 36, 48, and 60 months.

In some embodiments, a survival assessment is made at months 3, 6, 9, and 12 and then every 6 months thereafter following study discontinuation of completion of the adjuvant stage.

As used herein, the term “Disease-free survival (DFS) refers to the time span from the date of RC to the first recurrence of disease, post-RC, or death from any cause.

Thus, success of treatment may be defined as an increase in EFS, pCR, DFS and/or OS in patients treated with (i) neoadjuvant durvalumab and neoadjuvant chemotherapy compared to patients treated with neoadjuvant chemotherapy only; or (ii) perioperative durvalumab and neoadjuvant chemotherapy compared to patients treated with neoadjuvant chemotherapy only; or (iii) neoadjuvant durvalumab, neoadjuvant chemotherapy, and adjuvant durvalumab compared to patients treated with neoadjuvant chemotherapy only.

In some embodiments, the methods disclosed herein result in a 12-month event-free survival (EFS12) in at least about 76% of patients. This disclosure provides for methods of treating a patient having bladder cancer that results in a 24-month event-free survival (EFS24) in at least about 67% of patients. In some embodiments the methods disclosed herein results in about a 30% reduced risk of disease or death when compared to patients not receiving durvalumab as part of the neoadjuvant therapy. This disclosure provides for methods of treating a patient having bladder cancer that produces a pathological complete response (pCR) in more than 37% of patients. This disclosure provides for methods of treating a patient having bladder cancer that reduces the number of events resulting in an overall survival (OS) in more than 25% of patients.

Intent-to-treat (ITT) analysis set includes all randomized patients. Treatment groups are compared on the basis of randomized study treatment, regardless of the treatment actually received. Patients who were randomized but did not subsequently go on to receive study treatment are included in the analysis in the treatment group to which they were randomized.

Secondary endpoints include, but are not limited to, metastasis-free survival and disease-specific survival.

As used herein, the term “metastasis-free survival” refers to the time from the date of randomization until the first recognition of distant metastases or death, whichever occurred first.

As used herein, the term “disease-specific survival” refers to the time from date of randomization until death from bladder cancer.

The terms “administration” or “administering” as used herein refer to providing, contacting, and/or delivering a compound or compounds by any appropriate route to achieve the desired effect. Administration may include, but is not limited to, oral, sublingual, parenteral (e.g., intravenous, subcutaneous, intracutaneous, intramuscular, intraarticular, intraarterial, intrasynovial, intrasternal, intrathecal, intralesional, or intracranial injection), transdermal, topical, buccal, rectal, vaginal, nasal, ophthalmic, via inhalation, and implants.

It is to be understood that the particular aspects of the specification are described herein are not limited to specific embodiments presented and can vary. It also will be understood that the terminology used herein is for the purpose of describing particular aspects only and, unless specifically defined herein, is not intended to be limiting. Moreover, particular embodiments disclosed herein can be combined with other embodiments disclosed herein, as would be recognized by a skilled person, without limitation.

Without limiting the disclosure, a number of embodiments of the disclosure are described below for purpose of illustration.

### EXAMPLES

The Examples that follow are illustrative of specific embodiments of the disclosure, and various uses thereof. They are set forth for explanatory purposes only and should not be construed as limiting the scope of the invention in any way.

### Example 1: Neoadjuvant/Adjuvant Durvalumab in Combination with Gemcitabine and Cisplatin for the Treatment of Patients with Muscle-Invasive Bladder Cancer

The NIAGARA (NCT03732677) study described herein is a phase III, randomized, open-label, multi-center global study of durvalumab in combination with neoadjuvant gemcitabine+cisplatin before cystectomy (surgery to remove the bladder) followed by durvalumab as adjuvant monotherapy in patients with histologically or cytologically documented, resectable muscle-invasive bladder cancer and transitional cell carcinoma of the urothelium.

In this Phase III study the effectiveness of PD-L1 inhibition through exposure to neoadjuvant durvalumab, in combination with Gemcitabine+Cisplatin (G+C), followed, by the administration of durvalumab alone after radical cystectomy surgery was assessed as a therapy for increasing event free survival, pathological complete response and overall survival in patients with MIBC by preventing the MIBC tumor cells (TCs) from evading immune-mediated antitumor response.

### Eligible Patients

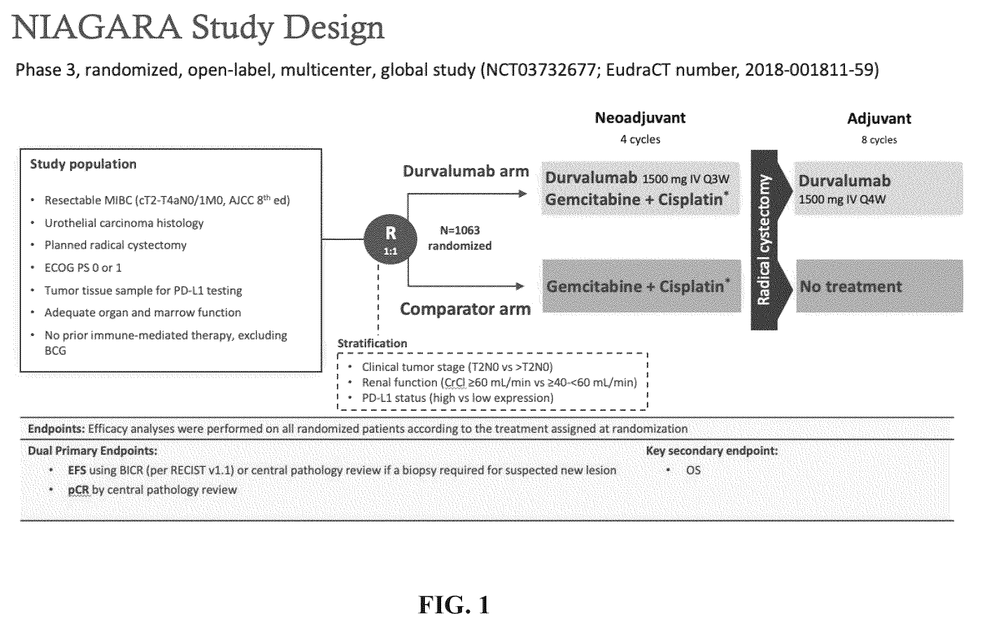

The study enrolled 1530 patients and randomized 1063 of those the patients in a 1:1 ratio to receive durvalumab+Gemcitabine+Cisplatin (D+G+C) combination therapy every three weeks (q3w) (Arm 1) or G+C combination therapy q3w (Arm 2) for 4 cycles of neoadjuvant chemotherapy prior to radical cystectomy. Patients started adjuvant durvalumab or placebo administration following surgery as soon as clinically feasible and within 42-120 days following surgery from surgery. Following radical cystectomy and during adjuvant therapy, patients in Arm 1 received durvalumab monotherapy every 4 weeks (q4w) for 8 additional cycles, and patients in Arm 2 received no adjuvant treatment. A summary of the study design is shown in FIG. 1.

Randomization was stratified by clinical tumor stage T2NO versus>T2NO (including T2N1, T3 and T4a) to reflect prognostic stage II versus IIIa, respectively; renal function (adequate renal function versus borderline renal function), and PD-L1 status (high versus low/negative, based on Ventana assay). Patients provided a tumor tissue sample at screening to determine PD-L1 status for stratification.

Eligible patients had histologically or cytologically documented muscle-invasive TCC (also known as UC) of the bladder, this includes patients with transitional cell and mixed transitional/non-transitional cell histologies (adenocarcinoma, squamous cell)/variant transitional (e.g., micropapillary, plasmacytoid, sarcomatoid, nested variant, lymphoepitheliod, nested variant) histologies. Eligible patients had histologically or cytologically documented, clinical stage T2-T4aN0/1M0 (according to AJCC Cancer Staging Manual, 8th edition) muscle-invasive TCC of the bladder. Patients were eligible for cisplatin-based chemotherapy, but had not previously been treated with chemotherapy or immunotherapy for MIBC. Patients were eligible to undergo surgical removal of the bladder.

Patients further had an Eastern Cooperative Oncology Group (ECOG) performance status (PS) of 0 or 1 at enrollment, a provided tumor sample with tumor PD-L1 status confirmed, and demonstrated adequate organ, marrow function, and body weight.

### Treatments and Treatment Duration

Patients received a combination therapy comprising 1500 mg durvalumab or placebo via intravenous (IV) infusion q3w for up to a maximum of 4 cycles prior to surgery and a platinum therapy consisting of cisplatin and gemcitabine, the dose dependent on renal function. Patients having a creatinine clearance rate of about ≥60 mL/min, were administered cisplatin at a dose of about 70 mg/m² on day 1 of each cycle; and (b) gemcitabine at a dose of about 1000 mg/m² on days 1 and 8 of each cycle every 3 weeks (q3w) for a total of four cycles. Patients having a creatinine clearance rate of about ≥40 mL/min to <60 mL/min, were administered cisplatin at a dose of about 70 mg/m² on day 1 of each cycle; and (b) gemcitabine at a dose of about 1000 mg/m² on days 1 and 8 of each cycle every 3 weeks (q3w) for a total of four cycles. The treatment schedule is summarized in Tables 1 and 2, and FIG. 1.

A radical cystectomy was performed as soon as possible after completion of fourth administration of the combination therapy. The radical cystectomy was performed between 56 and 70 days following the last administration of the combination therapy. Male patients underwent radial cytoprostatectomy and urinary diversion along with bilateral pelvic lymph node dissection. Female patients underwent radical cystectomy. Elective hysterectomy and bilateral salpingectomy was optionally performed if no gross evidence of oncologic involvement was noted by the surgeon. Oophorectomy was omitted if clinically indicated. A urinary diversion creation was established via an ileal conduit, ileal neobladder, Indiana Pouch, or as per local practice. Bilateral pelvic lymph node dissection included following template to include the 1) external iliac, 2) obturator, and 3) internal iliac nodes, and 4) common iliac nodes to the level of the ureteric crossing and included a lymph node classified as cN1 at baseline if one was present. Removal of other nodes is per the surgeon's discretion. The boundaries of the template were the circumflex iliac vein and Cloquet's node, laterally by the genitofemoral nerve, medially by the bladder, posteriorly by the obturator fossa, and proximally to the ureter. Radical cystectomy was mandatory study procedure for all patients unless a patient is only able to undergo a partial cystectomy.

TABLE 1
Treatment Schedule

| | | | | | | Adjuvant Therapy |
|---|---|---|---|---|---|---|
| | | | | | | 1 cycle = 4 weeks |
| | Neoadjuvant Therapy | | | | | (28 days) |
| | 1 cycle = 3 weeks | | | | Radical | Treatment will |
| | (21 days) | | | | Cystectomy ~56 | start 42-120 days |
| Treatment | Cycle 1 | Cycle 2 | Cycle 3 | Cycle 4 | days | following radical |
| Arm | Week 1 | Week 4 | Week 7 | Week 10 | (8 weeks) | cystectomy |

| | | | | | |
|---|---|---|---|---|---|
| Durva + G + C | Durva + | Durva + | Durva + | Durva + | Durva × 8 cycles |
| (Arm 1) | G + C | G + C | G + C | G + C | |
| G + C | G + C | G + C | G + C | G + C | No adjuvant |
| (Arm 2) | | | | | treatment |

Durva = Durvalumab; G + C = Gemcitabine and cisplatin.
Note:
If there is a dosing delay for neoadjuvant chemotherapy, durvalumab doses should be held to ensure that dosing is administered on Day 1 of each cycle.

TABLE 2
Treatment Scheme

| | | | |
|---|---|---|---|
| Neoadjuvant | Adequate Renal | Arm 1 | Day 1: durvalumab 1500 mg IV, cisplatin |
| Therapy | Function | | 70 mg/m², gemcitabine 1000 mg/m²; |
| | (CrCl > 60 mL/min) | | Day 8: gemcitabine 1000 mg/m²; every |
| | | | 21 days for 4 cycles. |
| | | Arm 2 | Day 1: cisplatin 70 mg/m², gemcitabine |
| | | | 1000 mg/m². |
| | | | Day 8: gemcitabine 1000 mg/m²; every |
| | | | 21 days for 4 cycles. |
| Adjuvant | Borderline Renal | Arm 1 | Day 1: durvalumab 1500 mg IV, cisplatin |
| Therapy | Function | | 35 mg/m², gemcitabine 1000 mg/m²; |
| | (CrCl > 40 mL/min | | Day 8: gemcitabine 1000 mg/m²; |
| | to <60 mL/min) | | cisplatin 35 mg/m²; every 21 days for 4 |
| | | | cycles |
| | | Arm 2 | Day 1: cisplatin 35 mg/m², gemcitabine |
| | | | 1000 mg/m². |
| | | | Day 8: gemcitabine 1000 mg/m²; |
| | | | cisplatin 35 mg/m²; every 21 days for 4 |
| | | | cycles. |
| | | Arm 1 | Day 1: durvalumab 1500 mg IV; every |
| | | | 28 days for 8 cycles. |
| | | Arm 2 | No adjuvant treatment. |

### Endpoints and Assessment

Dual primary efficacy endpoints evaluated in this study were event-free survival (EFS; per blinded independent central review [BICR]) and pathological complete response (pCR; evaluated centrally). Therefore, if either the EFS endpoint or pCR endpoint was achieved, the study was considered a success.

Secondary endpoints included overall survival (OS), and evaluation of the primary outcome in the PD-L1 high subgroup; pharmacokinetics and immunogenicity; patient-reported outcomes; and safety. In accordance with FDA guidance on clinical trial endpoints for the approval of cancer drugs and biologics (FDA 2018), EFS was defined as the time from randomization to the first of any of the following events: (1) the first recurrence of disease post-radical cystectomy, or (2) death due to any cause, or (3) time of first documented progression in patients who were precluded from radical cystectomy, or (4) time of expected surgery in patients who refuse to undergo a radical surgery, or failure to undergo a radical cystectomy in participants with residual disease. Additional secondary endpoints included metastasis-free survival and disease-specific survival. An exploratory post hoc analysis of EFS and OS in patients with pCR versus without pCR (non-pCR) was also performed.

Primary tumors and sampled lymph nodes were assessed for pathological response to neoadjuvant treatment by central pathology review using recommendations from the IASLC. To be eligible for pathological assessment, patients should have had three cycles of neoadjuvant study treatment per the protocol; patients who were not eligible for assessment (including those with R2 resection margins by local assessment) or who did not have a surgical specimen were classified as non-responders. pCR was defined as a lack of any viable tumor cells after complete evaluation of the resected lung cancer specimen and all sampled regional lymph nodes, and MPR was defined as ≤10% of viable tumor cells in the lung primary tumor (Travis et al., 2020); Hellmann et al., 2014; Cottrell et al., 2018).

DFS was determined as a secondary endpoint and assessed in all patients who received a RC and had a post-RC baseline scan, regardless of pathological high-risk features. DFS subgroup analysis and an exploratory post hoc analysis of DFS in patients with pCR vs patients without pCR (non-pCR) was performed. Data cutoff for DFS analysis was April 2024.

### Study Assessments and Procedures

### Tumor Assessments

pCR efficacy assessment was accomplished by pathological review of radical cystectomy samples. EFS efficacy assessment were assessed by either RECIST 1.1 on imaging scans by pathology on biopsy samples if required.

### Investigator RECIST 1.1 Imaging Review

Radiological efficacy was assessed by RECIST 1.1. Efficacy was evaluated using two baseline assessments, one for the neoadjuvant phase and the other for the adjuvant phase. The first baseline scan was collected during pre-randomization screening (Day-28 to Day-1) for disease staging and provided the RECIST 1.1 baseline for the post-neoadjuvant follow-up scan prior to radical cystectomy surgery. The second baseline scan was collected 42 days (+2 weeks) after the radical cystectomy procedure and performed prior to the first adjuvant treatment.

If observable tumors were identified, a new selection of target and/or non-target lesions was recorded. Follow-up scans were performed at least 4 weeks later as an assessment using RECIST 1.1 criteria and every 12 weeks thereafter. Earlier scans were utilized to allow early confirmation of metastatic lesions. New lesions were evaluated pathologically at any time when feasible to confirm metastatic disease.

Adjuvant tumor assessments occurred every 12 weeks±7 days after radical cystectomy surgery for the first 24 months, then every 24 weeks±7 days for 36 months. Following the first 36 months, assessments occurred every 52 weeks (annually) thereafter until unequivocal progression, the end of study, death, study discontinuation, or Sponsor decision, whichever comes first.

### Adverse Events

Adverse Events (AEs), Serious Adverse Events (SAE), and immune-mediated adverse events (imAE) were collected from the time of the patient signing the informed consent form until follow-up period as completed (90 days after last dose of treatment (Arm 1) or adjuvant phase study visits (Arm 2). Tables 3 and 4 outline the variables collected for adverse events and severe adverse events.

TABLE 3
Adverse Event Data Collection

| | |
|---|---|
| 1. | AE (verbatim) |
| 2. | The date when the AE started and stopped |
| 3. | The maximum CTCAE grade reported |
| 4. | Changes in CTCAE grade (report only the maximum |
| | CTCAE grade for a calendar day) |
| 5. | Whether the AE is serious or not |
| 6. | Investigator causality rating against the IPs |
| | (yes or no) |
| 7. | Action taken with regard to IPs |
| 8. | Administration of treatment for the AE |
| 9. | Outcome |

TABLE 4
Serious Adverse Event Data Collection

| | |
|---|---|
| 1. | Date the AE met criteria for SAE |
| 2. | Date the investigator became aware of the SAE |
| 3. | Seriousness criteria |
| 4. | Date of hospitalization |
| 5. | Date of discharge |
| 6. | Probable cause of death |
| 7. | Date of death |
| 8. | Whether an autopsy was performed |
| 9. | Casualty assessment in relation to study procedure(s) |
| 10. | causality assessment in relation to other medication |
| 11. | Description of the SAE |

An adverse event of special interest (AESI) is one of scientific and medical interest specific to understanding of the IP and may require close monitoring. An AESI may be serious or non-serious. The rapid reporting of AESIs allows ongoing surveillance of these events to best characterize and understand them in association with the use of the IP.

AESIs for durvalumab include but are not limited to events with a potential inflammatory or immune-mediated mechanism and which may require more frequent monitoring and/or interventions such as steroids, immunosuppressants, and/or hormone replacement therapy. These AESIs are closely monitored in clinical studies with durvalumab monotherapy and combination therapy. An imAE is defined as an AESI that is associated with drug exposure, required the use of systemic steroids or other immunosuppressants and/or endocrine therapy for specific endocrine events, is consistent with an immune-mediated MOA, and where there is no clear alternate etiology.

Serologic, immunologic, and histologic (biopsy) data, as appropriate, were used to support an imAE diagnosis. Appropriate efforts were made to rule out neoplastic, infectious, metabolic, toxin, or other etiologic causes of the imAE.

AESIs/imAEs observed with anti PD-L1/PD-1 agents such as durvalumab include pneumonitis, hepatitis, diarrhea/colitis, intestinal perforation, endocrinopathies (hypo- and hyper-thyroidism, adrenal insufficiency, hypophysitis/hypopituitarism and Type 1 diabetes mellitus), nephritis, rash/dermatitis, myocarditis, myositis/polymyositis, pancreatitis and rare/less frequent imAEs including neuromuscular toxicities such as myasthenia gravis and Guillain-Barre syndrome.

Other inflammatory responses that are rare/less frequent with a potential immune-mediated etiology include, but are not limited to, pericarditis, sarcoidosis, uveitis, and other events involving the eye, skin, hematological, rheumatological events, vasculitis, non-infectious meningitis and non-infectious encephalitis. It is possible that events with an inflammatory or immune mediated mechanism could occur in nearly all organs.

In addition, infusion-related reactions and hypersensitivity/anaphylactic reactions with a different underlying pharmacological etiology are also considered AESIs.

### Results (DCO April 2024)

### Efficacy

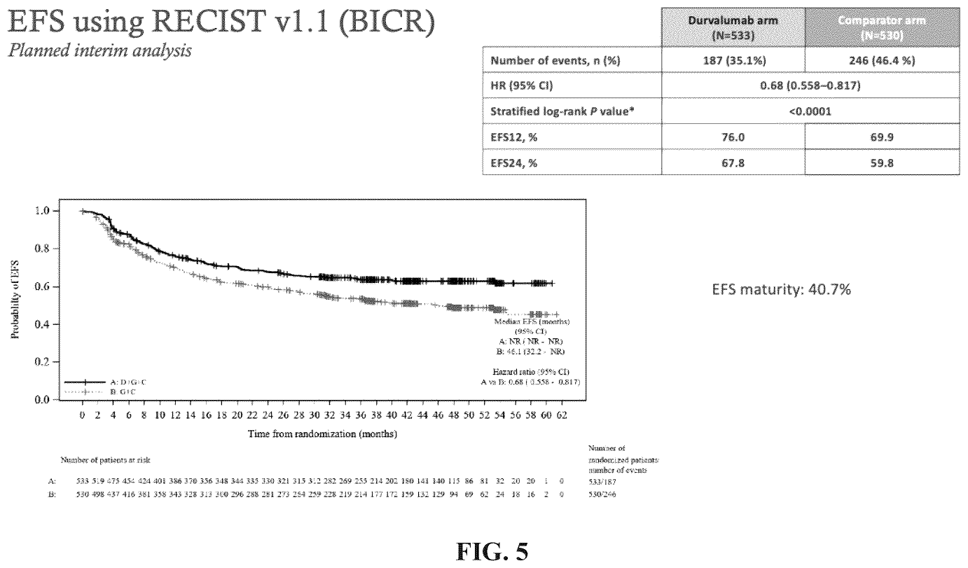

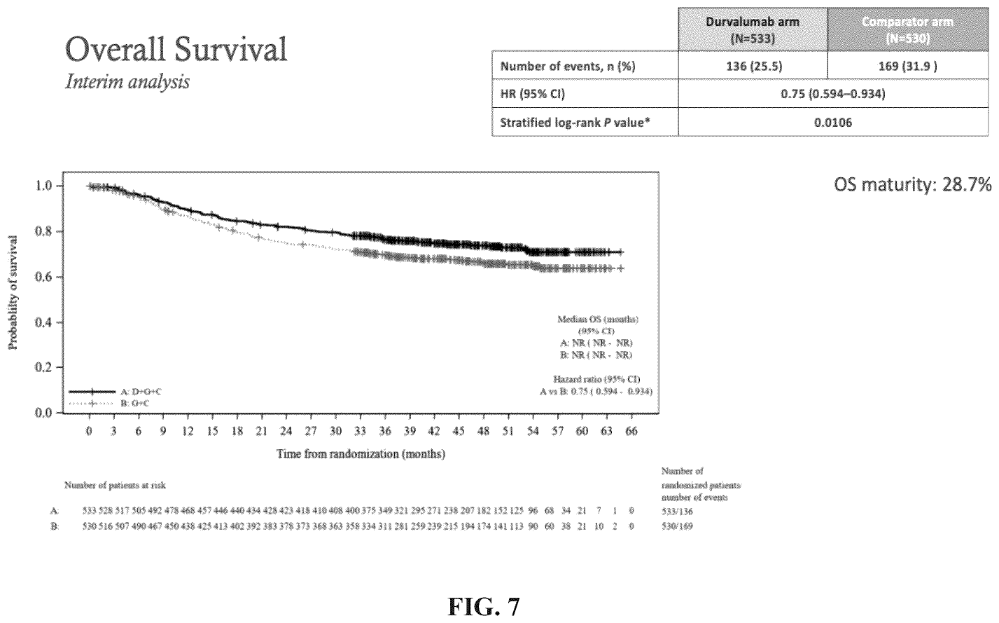

NIAGARA met the primary endpoint of EFS demonstrating a statistically significant improvement in the intention-to-treat (ITT) population for the durvalumab arm (Durvalumab+cisplatin+gemcitabine) vs the comparator arm (cisplatin+gemcitabine). Hazard Ratio (HR)=0.68, 95% CI 0.558-0.817, p<0.0001. See FIG. 5. After greater than 40 month of follow-up patients in the durvalumab arm had a 33% reduction in risk of developing a distant metastasis or dying (hazard ratio [HR] 0.67; 95% confidence interval [CI] 0.54-0.83; nominal p<0.001) and a 31% reduction in risk of death from bladder cancer (HR 0.69; 95% CI 0.52-0.91; nominal p=0.008) vs patients in the comparator arm. Thus, patients in the durvalumab arm had an improvement in OS versus the compactor arm, as evidenced by a 25% reduction in the relative risk of death from any cause (HR 0.75, 95% CI 0.594-0.934, p=0.0106). See FIG. 7.

### Metastasis Free Survival (ITT)

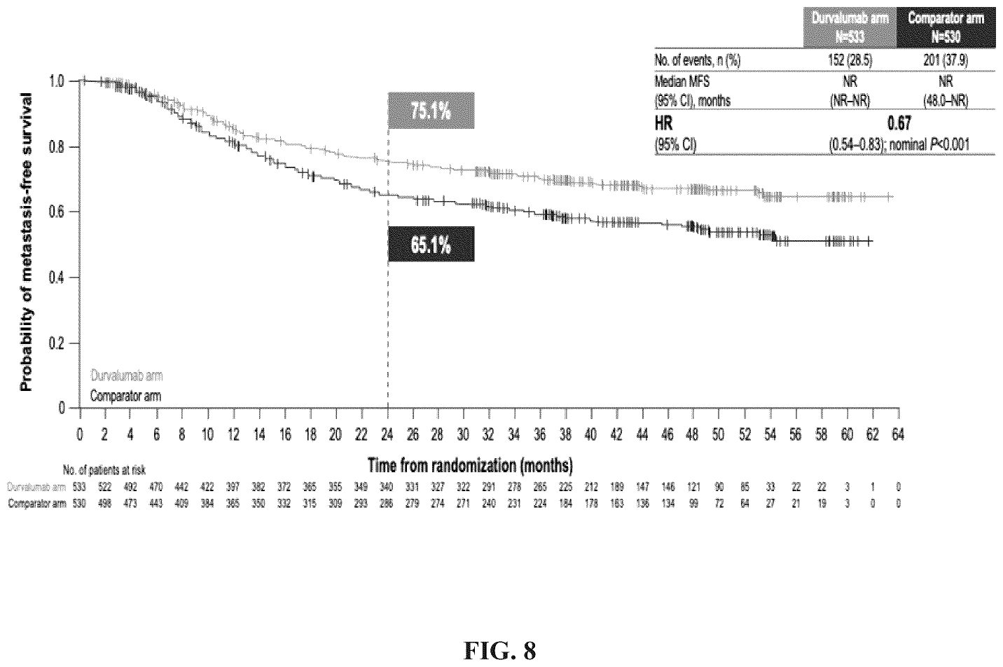

The secondary endpoint of metastasis-free survival was improved in the ITT population for the durvalumab arm (Durvalumab+cisplatin+gemcitabine) vs the comparator arm. Patients in the durvalumab arm had a 33% reduction in risk of developing a distant metastasis or dying (hazard ratio [HR] 0.67; 95% confidence interval [CI] 0.54-0.83; nominal p<0.001). The initial recognition of metastasis-free survival or distant metastases or death occurred in 152 of the 533 patients in the durvalumab arm (28.5%) and 201 of the 530 patients in the comparator arm (37.9%) with a hazard ratio of 0.67 (95% confidence interval of 0.54-0.83). The addition of durvalumab to neoadjuvant chemotherapy (NAC) resulted in a 75.1% probability of 24-month metastasis-free survival, compared with a 65.1% probability of 24-month metastasis-free survival in the patients in the comparator arm receiving only NAC (See FIG. 8).

### Disease Specific Survival (ITT)

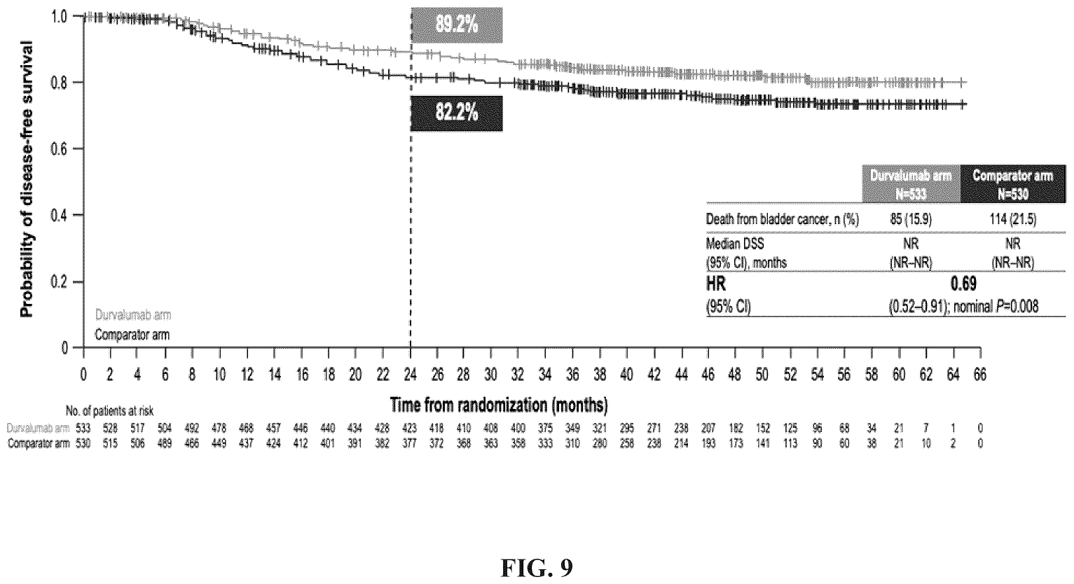

The secondary endpoint of disease-specific survival was improved in the ITT population for the durvalumab arm (Durvalumab+cisplatin+gemcitabine) vs the comparator arm (cisplatin+gemcitabine). Patients in the durvalumab arm had a 31% reduced risk of death from bladder cancer (Hazard Ratio (HR)=0.69, 95% CI 0.52-0.91, p<0.0001). More specifically, death from bladder cancer occurred in 85 of the 533 patients in the durvalumab arm (15.9%) and 114 of the 530 patients in the comparator arm. The addition of durvalumab to NAC increased the 24-month probability of disease-free survival to 89.2% from 82.2% in patients in the comparator arm receiving only NAC. Overall the administration of durvalumab with NAC reduced the risk of bladder cancer by 31% (see FIG. 9).

pCR

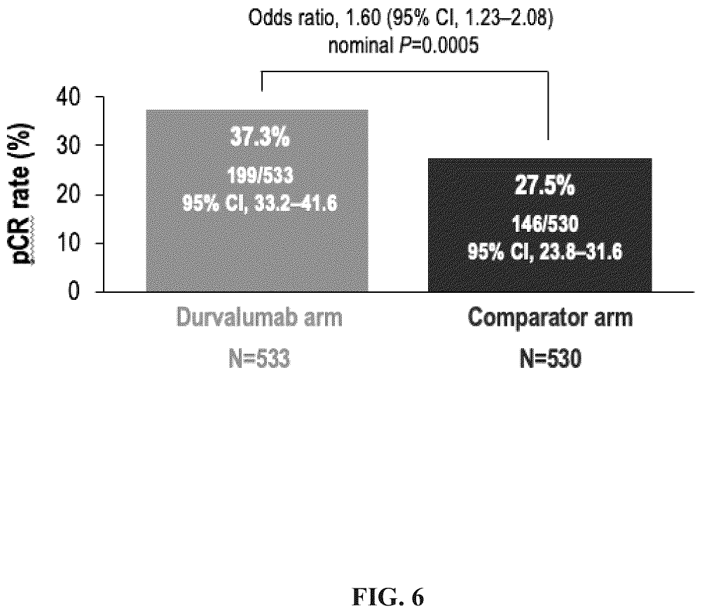

Perioperative durvalumab with neoadjuvant chemotherapy reduced the risk of developing metastases and death from bladder cancer. More specifically, an increased number of patients in the durvalumab arm (37.3%) vs comparator arm (27.5%) had pCR at the time of the radical cystectomy (odds ratio 1.60, nominal p=0.0005). Thus, patients receiving durvalumab, in addition to chemotherapy demonstrated an ˜10% in pCR. See FIG. 6. Patients with pCR derived greater benefit with durvalumab treatment versus those patients in the comparator arm (EFS HR 0.58; OS HR 0.72); a similar finding was observed for non-pCR patients (EFS HR 0.77; OS HR 0.84) (Table 5). Patients that achieved pCR and treated with durvalumab had a 42% reduction in relative risk of tumor growth, tumor spreading, not undergoing surgery or dying. Similarly, patients achieving pCR and treated with durvalumab were further characterized by increased overall survival, as evidenced by a 28% reduction in the relative risk of dying from any cause. Non-pCR patients treated with durvalumab demonstrated a 23% reduction in relative risk tumor growth, tumor spreading, not undergoing surgery or dying and a 16% reduction in the relative risk of dying from any cause. Thus, the exploratory analyses showed durvalumab improved EFS and OS in both pCR and non-pCR groups. The administration of durvalumab with NAC reduces the risk of metastases and death from bladder cancer thereby indicating perioperative durvalumab as a new standard treatment for patients with MIBC.

TABLE 5
Efficacy of Durvalumab Treatment

| | pCR | | Non-pCR | |
|---|---|---|---|---|
| | Durvalumab | Comparator | Durvalumab | Comparator |
| Treatment Arm | N = 199 | N = 146 | N = 334 | N = 384 |
| 24-month EFS | 92% | 86% | 53% | 50% |
| rate (95% CI) | (87-95) | (79-91) | (48-59) | (44-55) |
| HR (95% CI) | 0.58 | | 0.77 | |
| | (0.33-1.00) | | (0.63-0.95) | |
| 24-month OS | 96% | 91% | 74% | 69% |
| rate | (92-98) | (85-95) | (69-79) | (64-73) |

| | | |
|---|---|---|
| HR (95% CI) | 0.72 | 0.84 |
| | (0.37-1.43) | (0.66-1.07) |

EFS = Event Free Survival;
pCR = pathological complete response;
HR = Hazards Ratio

### Disease Free Survival (DFS)

A total of 165 of the 1063 patients randomized to the durvalumab arm and comparator arm had tumors with divergent differentiation or other histologic subtypes. Among patients that underwent RC (durvalumab arm=469 patients; comparator arm=441 patients), the pCR rates were increased in the durvalumab arm versus the comparator arm.

Patients in the durvalumab arm that underwent a radical cystectomy and completed a post-RC baseline scan had a 31% reduced risk of disease recurrence or death vs patients in the comparator arm (DFS hazard ratio [HR] 0.69; 95% CI 0.51-0.93; nominal P=0.014). The increased DFS in patients in the durvalumab arm was observed across subgroups, including in patients with divergent or other histologic subtypes (total n=103; HR 0.45 95% CI 0.19-0.99). Patients in the durvalumab arm derived greater DFS benefit versus the comparator arm in both the pCR (HR 0.58; 95% CI 0.33-1.00) and non-pCR (HR 0.82; 95% CI 0.64-1.05) groups. Perioperative durvalumab with neoadjuvant chemotherapy (NAC) reduced the risk of disease recurrence or death versus NAC alone in pts with MIBC, including those with and without pathologic high-risk features, who underwent RC.

The exploratory post hoc analysis showed that DFS favored patients receiving durvalumab in both the pCR and non-pCR groups. Collectively, these results further support the addition of perioperative durvalumab to NAC+RC as a potential new treatment option for MIBC.

### Safety

The overall safety profile is consistent with the known safety profile of durvalumab and chemotherapy. The addition of durvalumab to chemotherapy had no detrimental impact on backbone chemotherapy administration. AEs leading to treatment discontinuation in neoadjuvant phase were balanced between the two arms. The number of patients not undergoing radical surgery because of AEs due to neoadjuvant treatment was balanced in both arms. Rates of grade 3 or 4 AEs and AEs with outcome of death were similar between the two arms (See Table 6). The overall study period included AEs that occurred between the first dose of study treatment, and whichever occurred first: 1) 90 days after the last dose of treatment, surgery, or last adjuvant visit; 2) date of first dose of subsequent anti-cancer therapy; or 3) data cut-off date.

Immune-mediated AEs (imAEs) were low grade throughout the study. Overall, imAEs were reported in 21% (111/530) of patients in the durvalumab arm (grade 3/4; 3%) and 3% (16/526) of patients in the comparator arm (grade 3/4<1%). At data cutoff (DCO), imAEs were resolved in 41% (45/111) of patients in the durvalumab arm 44% (7/16) of patients in the comparator arm. The most common imAEs were hypothyroid events (10% durvalumab arm; 1% comparator arm) and hyperthyroid events (3% durvalumab arm; <1% comparator arm). Additional imAEs in the durvalumab group included dermatitis/rash, renal events, diarrhea/colitis, and pneumonitis, all of which were 2% or less in patients receiving durvalumab.

TABLE 6
Adverse Event Summary

| Overall study period (unless | Durvalumab arm | | Comparator arm | |
|---|---|---|---|---|
| otherwise stated) | (N = 530) | | (N = 526) | |
| Any-grade AEs, n (%) | 527 | (99.4) | 525 | (99.8) |
| Max grade 3 or 4 | 368 | (69.4) | 355 | (67.5) |
| SAEs | 326 | (61.5) | 287 | (54.6) |
| Outcome of death | 27 | (5.1) | 29 | (5.5) |
| Leading to discontinuation of study | 112 | (21) | 80 | (15) |
| treatment | | | | |
| Leading to discontinuation of | 50 | (9.4) | — | |
| neoadjuvant durvalumab | | | | |
| Leading to discontinuation of NAC | 72 | (13.6) | 80 | (15.2) |
| Leading to patient not undergoing | 6 | (1.1) | 7 | (1.3) |
| RC | | | | |
| Leading to delay in surgery* | 9 | (2.0) | 6 | (1.0) |
| Leading to discontinuation of | 30 | (7.8) | — | |
| adjuvant durvalumab | | | | |
| Any-grade AEs possibly related | 502 | (94.7) | 487 | (92.6) |
| to any treatment, n (%) | | | | |
| Max grade 3 or 4 | 215 | (40.6) | 215 | (40.9) |
| Outcome of death | 3 | (0.6) | 3 | (0.6) |

*Defined as outside recommended timeframe for RC of 56 days after last dose of NAC AE, adverse event; NAC, neoadjuvant chemotherapy; RC, radical cystectomy.

The most common AEs (≥15%) were nausea, anemia, constipation, fatigue, urinary tract infection, decreased appetite, neutropenia, pyrexia, diarrhea (See Table 7). AEs were balanced between arms and are mostly attributed to chemotherapy and disease under investigation.

TABLE 7
Most Common AEs (Safety Analysis Set)*

| | Durvalumab | | Comparator | |
|---|---|---|---|---|
| | Arm | | Arm | |
| Adverse Event | (N = 530) | | (N = 526) | |
| Patients with any AE (%) | 527 | (99.4) | 525 | (99.8) |
| Nausea | 284 | (53.6) | 255 | (48.5) |
| Anemia | 205 | (38.7) | 213 | (40.5) |
| Constipation | 205 | (38.7) | 203 | (38.6) |
| Fatigue | 191 | (36.0) | 169 | (32.1) |
| Urinary tract infection | 159 | (30.0) | 153 | (29.1) |
| Decreased appetite | 141 | (26.6) | 131 | (24.9) |
| Neutropenia | 137 | (25.8) | 165 | (31.4) |
| Pyrexia | 110 | (20.8) | 87 | (16.5) |
| Diarrhea | 109 | (20.6) | 74 | (14.1) |
| Vomiting | 102 | (19.2) | 97 | (18.4) |
| Blood creatinine increased | 98 | (18.5) | 77 | (14.6) |
| Asthenia | 93 | (17.5) | 96 | (18.3) |
| Neutrophil count | 81 | (15.3) | 74 | (14.1) |
| decreased | | | | |
| Pruritis | 80 | (15.1) | 38 | (7.2) |

*Frequency of >15% Overall Period

### BIBLIOGRAPHY

1. AJCC Cancer Staging Manual (Amin et al., eds., 8th Edition, 2017).
2. Alexandrov et al., “Signatures of mutational processes in human cancer,” Nature 500 (7463): 415-21 (2013).
3. Babjuk et al., “Guidelines on non-muscle-invasive bladder cancer (Ta, T1, and CIS)” (EAU Guidelines Office, Arnhem, The Netherlands, European Association of Urology, 2014).
4. Boccardo et al., “Adjuvant chemotherapy of bladder cancer,” Ann. Oncol. 17 (Suppl. 5): v129-32 (2006).
5. Burger et al., “Epidemiology and Risk Factors of Urothelial Bladder Cancer,” Eur. Urol. 63 (2): 234-41 (2013).
6. Chang et al., “Treatment of nonmetastatic muscle-invasive bladder cancer: AUA/ASCO/ASTRO/SUO guideline,” American Urological Association 1-42 (2017).
7. Cottrell et al., “Pathologic features of response to neoadjuvant anti-PD-1 in resected non-small-cell lung carcinoma: a proposal for quantitative immune-related pathologic response criteria (irPRC),” Ann. Oncol. 29 (8): 1853-60 (2018).
8. Dindo et al., “Classification of surgical complications: a new proposal with evaluation in a cohort of 6336 patients results of a survey,” Ann. Surg. 240 (2): 205-13 (2004).
9. Ferlay et al., “Cancer incidence and mortality worldwide: Sources, methods and major patterns in GLOBOCAN 2012,” Int. J. Cancer 136 (5): E359-86 (2015).
10. Grossman et al., “Neoadjuvant chemotherapy plus cystectomy compared with cystectomy alone for locally advanced bladder cancer,” N. Engl. J. Med. 349 (9): 859-66 (2003).
11. Hellmann et al., “Pathological response after neoadjuvant chemotherapy in resectable non-small-cell lung cancers: proposal for the use of major pathological response as a surrogate endpoint,” Lancet Oncol. 15 (1): e42-50 (2014).
12. Inman et al., “PD-L1 (B7-H1) expression by urothelial carcinoma of the bladder and BCG-induced granulomata: associations with localized stage progression,” Cancer 109 (8): 1499-505 (2007).
13. Kim et al., “Adjuvant chemotherapy for muscle-invasive bladder cancer: a systematic review and network meta-analysis of randomized clinical trials,” Oncotarget 8 (46): 81204-14 (2017).
14. Kodumudi et al., “A novel chemoimmunomodulating property of docetaxel: suppression of myeloid-derived suppressor cells in tumor bearers,” Clin. Cancer Res. 16 (18): 4583-94 (2010).
15. Kroemer et al., “Immunogenic cell death in cancer therapy,” Annu. Rev. Immunol. 31:51-72 (2013).
16. Langer et al., “Carboplatin and pemetrexed with or without pembrolizumab for advanced, non-squamous nonsmall-cell lung cancer: a randomized, phase 2 cohort of the open-label KEYNOTE-021 study,” Lancet Oncol. 17 (11): 1497-508 (2016).
17. Leow et al., “Adjuvant chemotherapy for muscle-invasive bladder cancer: a 2013 updated systematic review and metaanalysis of randomized trials,” Eur. Urol. 66 (1): 42-54 (2014).
18. Liu et al., “Pre-treatment with chemotherapy can enhance the antigenicity and immunogenicity of tumours by promoting adaptive immune responses,” Br. J. Cancer 102 (1): 115-23 (2010).
19. Mellman et al., “Cancer immunotherapy comes of age,” Nature 480 (7378): 480-89 (2011).
20. Necchi et al., “Preoperative pembrolizumab (pembro) before radical cystectomy (RC) for muscle-invasive urothelial bladder carcinoma (MIUC): Interim clinical and biomarker findings from the phase 2 PURE-01 study,” J. Clin. Oncol. 36 (15_suppl): 4507 (2018).
21. Powles et al., “MPDL3280A (anti-PD-L1) treatment leads to clinical activity in metastatic bladder cancer,” Nature 515 (7528): 558-62 (2014).
22. Sonpavde et al., “Quality of pathologic response and surgery correlate with survival for completely resected bladder cancer following neoadjuvant chemotherapy,” Cancer 115 (18): 4104-09 (2009).
23. Torre et al., “Global cancer statistics, 2012,” CA Cancer J. Clin. 65 (2): 87-108 (2015).
24. Travis et al., “IASLC Multidisciplinary Recommendations for Pathologic Assessment of Lung Cancer Resection Specimens After Neoadjuvant Therapy,” J. Thorac. Oncol. 15 (5): 709-40 (2020).
25. Witjes et al., “EAU Guidelines on muscle-invasive and metastatic bladder cancer,” European Association of Urology (EAU) Guidelines (2016).
26. Zhang et al., “Anti-tumor immunotherapy by blockade of the PD-1/PD-L1 pathway with recombinant human PD-1-IgV,” Cytotherapy 10 (7): 711-19 (2008).

What is claimed is:

1: A method of treating bladder cancer in a patient in need thereof, the method comprising:
(a) administering to the patient an anti-PD-L1 antibody or antigen binding fragment thereof and one or more platinum-based chemotherapies; and
(b) performing a radical cystectomy.

2: The method of claim 1, wherein the anti-PD-L1 antibody or antigen binding fragment thereof and one or more platinum-based chemotherapies are administered about every 2 to 4 weeks (Q2W-Q4W) for about 3 to 6 cycles.

3: The method of either claim 1 or claim 2, wherein the anti-PD-L1 antibody or antigen binding fragment thereof and one or more platinum-based chemotherapies are administered about every 3 weeks (Q3W) for about 4 cycles.

4: The method of any one of claims 1-3, wherein the anti-PD-L1 antibody or antigen binding fragment thereof is durvalumab.

5: The method of any one of claims 1-4, wherein the anti-PD-L1 antibody or antigen binding fragment thereof comprises about 500-2000 mg of durvalumab.

6: The method of claim 5, wherein the anti-PD-L1 antibody or antigen binding fragment thereof comprises about 1500 mg of durvalumab.

7: The method of any one of claims 1-4, wherein the anti-PD-L1 antibody or antigen binding fragment thereof comprises about 20 mg/kg of durvalumab.

8: The method of any one of claims 1-7, further comprising administering to the patient an adjuvant therapy comprising an anti-PD1-L1 antibody or antigen binding fragment thereof following the radical cystectomy.

9: The method of claim 8, wherein the adjuvant therapy comprises about 500-2000 mg of durvalumab.

10: The method of claim 9, wherein the adjuvant therapy comprises about 1500 mg of durvalumab.

11: The method of claim 8, wherein the adjuvant therapy comprises about 1500 mg of durvalumab administered to the patient about every 2 to 4 weeks (Q2W-Q4W) for up to about 12 cycles.

12: The method of any one of claims 1-7, wherein the adjuvant therapy comprises about 20 mg/kg of durvalumab.

13: The method of either claim 11 or claim 12, wherein the adjuvant therapy is administered to the patient about every 4 weeks (Q4W) for up to about 8 cycles.

14: A method of treating bladder cancer in a patient in need thereof, the method comprising:
(a) administering to the patient about 1500 mg of durvalumab and one or more platinum-based chemotherapies about every 3 weeks (Q3W) for about 4 cycles;
(b) performing a radical cystectomy; and
(c) administering to the patient an adjuvant therapy comprising about 1500 mg of durvalumab about every 4 weeks (Q4W) for about 8 cycles.

15: The method of any one of claims 1-14, wherein the one or more platinum-based chemotherapies are afatinib, bevacizumab, carboplatin, cetuximab, cisplatin, doxorubicin, erlotinib, gemcitabine, methotrexate, nedaplatin, oxaliplatin, paclitaxel, pemetrexed, emurafenib, and/or vinblastine.

16: The method of claim 15, wherein the one or more platinum-based chemotherapies are carboplatin and gemcitabine.

17: The method of any one of claims 1-16, wherein the one or more platinum-based chemotherapies are (a) cisplatin administered to the patient at a dose of about 10-100 mg/m² Q3W; and (b) gemcitabine administered to the patient at a dose of about 500-1500 mg/m² Q3W.

18: The method of claim 17, wherein, if the patient has a creatinine clearance of about ≥60 mL/min, the one or more platinum-based chemotherapies are (a) cisplatin administered to the patient at a dose of about 70 mg/m² on day 1 of each cycle; and (b) gemcitabine administered to the patient at a dose of about 1000 mg/m² on days 1 and 8 of each cycle.

19: The method of claim 17, wherein, if the patient has a creatinine clearance of about ≥40 mL/min to <60 mL/min, the one or more platinum-based chemotherapies are (a) cisplatin administered to the patient at a dose of about 35 mg/m² on days 1 and 8 of each cycle; and (b) gemcitabine administered to the patient at a dose of about 1000 mg/m² on days 1 and 8 of each cycle.

20: A method of treating bladder cancer in a patient in need thereof, the method comprising:
(a) administering to the patient:
(i) about 1500 mg of durvalumab;
(ii) gemcitabine at a dose of about 1000 mg/m² on days 1 and 8 of each cycle;
(iii) cisplatin at a dose of about 70 mg/m² on day 1 of each cycle if the patient has a creatinine clearance of about ≥60 mL/min or at a dose of about 35 mg/m² on days 1 and 8 of each cycle if the patient has a creatinine clearance of about ≥40 mL/min to <60 mL/min;
wherein the durvalumab, gemcitabine, and cisplatin are administered to the patient about every 3 weeks (Q3W) for 4 cycles;
(b) performing a radical cystectomy; and
(c) administering to the patient an adjuvant therapy comprising about 1500 mg of durvalumab about every 4 weeks (Q4W) for up to 8 cycles.

21: The method of any one of claims 1-20, wherein the anti-PD-L1 antibody or antigen binding fragment thereof is administered to the patient prior to administration of the one or more platinum-based chemotherapies.

22: The method of any one of claims 1-20, wherein the anti-PD-L1 antibody or antigen binding fragment thereof is administered to the patient at the same time as administration of the one or more platinum-based chemotherapies.

23: The method of any one of claims 1-22, wherein the radical cystectomy is performed about 14 days to about 70 days following administration of 4 cycles of the anti-PD-L1 antibody or antigen binding fragment thereof and one or more platinum-based chemotherapies.

24: The method of claim 23, wherein the radical cystectomy is preformed about 56 days following administration of 4 cycles of the anti-PD-L1 antibody or antigen binding fragment thereof and one or more platinum-based chemotherapies.

25: The method of any one of claim 8-14 or 20, wherein the adjuvant therapy comprises about 1500 mg of durvalumab administered to the patient every four weeks (Q4W) for up to about 32 weeks.

26: The method of any one of claim 8-14 or 20, wherein the adjuvant therapy is administered to the patient about 28 days to about 150 days following the radical cystectomy.

27: The method of claim 26, wherein the adjuvant therapy is administered to the patient about 42 days to about 120 days following the radical cystectomy.

28: The method of any one of claims 1-27, wherein the bladder cancer is muscle-invasive bladder cancer (MIBC) or transitional cell carcinoma (TCC) of the urothelium.

29: The method of claim 28, wherein the MIBC is resectable MIBC.

30: The method of any one of claims 1-29, wherein the patient has not received prior systemic chemotherapy or immunotherapy for treatment of MIBC.

31: A combination therapy for use in a method of treating a patient with bladder cancer, the method comprising (a) administering to the patient a combination therapy comprising an anti-PD-L1 antibody or antigen binding fragment thereof and one or more platinum-based chemotherapies; and (b) performing a radical cystectomy.

32: The combination therapy for the use of claim 31, wherein the anti-PD-L1 antibody or antigen binding fragment thereof and one or more platinum-based chemotherapies are administered about every 2 to 4 weeks (Q2W-Q4W) for about 3 to 6 cycles.

33: The combination therapy for the use of either claim 31 or claim 32 wherein the anti-PD-L1 antibody or antigen binding fragment thereof and one or more platinum-based chemotherapies are administered about every 3 weeks (Q3W) for about 4 cycles.

34: The combination therapy for the use of any one of claims 31-33, wherein the anti-PD-L1 antibody or antigen binding fragment thereof is durvalumab.

35: The combination therapy for the use of any one of claims 31-34, wherein the anti-PD-L1 antibody or antigen binding fragment comprises about 500-2000 mg of durvalumab.

36: The combination therapy for the use of claim 35, wherein the anti-PD-L1 antibody or antigen binding fragment comprises about 1500 mg of durvalumab.

37: The combination therapy for the use of any one of claims 31-34, wherein the anti-PD-L1 antibody or antigen binding fragment thereof comprises about 20 mg/kg of durvalumab.

38: The combination therapy for the use of claim 31, further comprising administering an adjuvant therapy comprising an anti-PD1-L1 antibody or antigen binding fragment thereof.

39: The combination therapy for the use of claim 38, wherein the adjuvant therapy comprises about 500-2000 mg of durvalumab.

40: The combination therapy for the use of claim 39, wherein the adjuvant therapy comprises about 1500 mg of durvalumab.

41: The combination therapy for the use of claim 38, wherein the adjuvant therapy comprises about 1500 mg of durvalumab administered to the patient every 2 to 4 weeks (Q2W-A4W) for up to about 12 cycles.

42: The combination therapy for the use of claim 38, wherein the adjuvant therapy comprises about 20 mg/kg of durvalumab.

43: The combination therapy for the use of claim 41 or claim 42, wherein the adjuvant therapy is administered to the patient about every 4 weeks (Q4W) for up to about 8 cycles.

44: A combination therapy for use in a method of treating a patient with bladder cancer, the method comprising:
(a) administering about 1500 mg of durvalumab and one or more platinum-based chemotherapies, wherein the durvalumab and one or more platinum-based chemotherapies are administered to the patient about every 3 weeks (Q3W) for about 4 cycles;
(b) performing a radical cystectomy; and
(c) administering an adjuvant therapy comprising about 1500 mg of durvalumab, wherein the adjuvant therapy is administered to the patient about every 4 weeks (Q4W) for up to about 8 cycles.

45: The combination therapy for the use of any one of claims 31-44, wherein the one or more platinum-based chemotherapies are afatinib, bevacizumab, carboplatin, cetuximab, cisplatin, doxorubicin, emurafenib, erlotinib, gemcitabine, methotrexate, nedaplatin, oxaliplatin, paclitaxel, pemetrexed, and/or vinblastine.

46: The combination therapy for the use of claim 45, wherein the one or more platinum-based chemotherapies are cisplatin and gemcitabine.

47: The combination therapy for the use of any one of claims 31-46, wherein the one or more platinum-based chemotherapies are (a) cisplatin administered to the patient at a dose of about 10-100 mg/m² Q3W and (b) gemcitabine administered to the patient at a dose of about 500-1500 mg/m² Q3W.

48: The combination therapy for the use of claim 47, wherein, if the patient has a creatinine clearance of about ≥60 mL/min, the one or more platinum-based chemotherapies are (a) cisplatin administered to the patient at a dose of about 70 mg/m² on day 1 of each cycle; and (b) gemcitabine administered to the patient at a dose of about 1000 mg/m² on days 1 and 8 of each cycle.

49: The combination therapy for the use of claim 47, wherein, if the patient has a creatinine clearance of about ≥40 mL/min to <60 mL/min, the one or more platinum-based chemotherapies are (a) cisplatin administered to the patient at a dose of about 35 mg/m² on days 1 and 8 of each cycle; and (b) gemcitabine administered to the patient at a dose of about 1000 mg/m² on days 1 and 8 of each cycle.

50: A combination therapy for use in treating a patient with bladder cancer, the method comprising administering about 1500 mg of durvalumab, gemcitabine at a dose of about 1000 mg/m², and cisplatin; wherein the cisplatin is administered to the patient at a dose of about 70 mg/m² if the patient has a creatinine clearance of ≥60 mL/min or at a dose of about 35 mg/m² if the patient has a creatinine clearance of ≥40 mL/min to <60 mL/min; and
wherein the durvalumab, gemcitabine, and cisplatin are administered to the patient about every 3 weeks (Q3W) for 4 cycles and performing a radical cystectomy.

51: The combination therapy for the use of any one of claims 31-50, wherein the anti-PD-L1 antibody or antigen binding fragment thereof is administered to the patient prior to administration of the one or more platinum-based chemotherapies.

52: The combination therapy for the use of any one of claims 31-50, wherein the anti-PD-L1 antibody or antigen binding fragment thereof is administered to the patient at the same time as administration of the one or more platinum-based chemotherapies.

53: The combination therapy for the use of claim 31 or claim 44, wherein the radical cystectomy is performed about 14 days to about 70 days following administration of 4 cycles of the anti-PD-L1 antibody or antigen binding fragment thereof and one or more platinum-based chemotherapies.

54: The combination therapy of claim 53, wherein the radical cystectomy is preformed about 56 days following administration of 4 cycles of the anti-PD-L1 antibody or antigen binding fragment thereof and one or more platinum-based chemotherapies.

55: The combination therapy for the use of any one of claim 38 or 44, wherein the adjuvant therapy comprises about 1500 mg of durvalumab administered to the patient every 4 weeks (Q4W) for up to about 8 cycles.

56: The combination therapy for the use of any one of claims 38-44, wherein the adjuvant therapy is administered to the patient about 28 days to about 150 days following a radical cystectomy.

57: The combination therapy for the use of claim 56, wherein the adjuvant therapy is administered to the patient about 42 days to about 120 days following the radical cystectomy.

58: The combination therapy for the use of any one of claims 31-57, wherein the bladder cancer is muscle-invasive bladder cancer (MIBC) or transitional cell carcinoma (TCC) of the urothelium.

59: The combination therapy for the use of claim 58, wherein the MIBC is resectable MIBC.

60: Use of a combination therapy comprising an anti-PD-L1 antibody or antigen binding fragment thereof and one or more platinum-based chemotherapies for the manufacture of a medicament for treating bladder cancer in a patient before the patient receives a radical cystectomy.

61: The use of claim 60, wherein the medicament is administered to the patient about every 2 to 4 weeks (Q2W-Q4W) for about 3 to 6 cycles.

62: The use of either claim 60 or claim 61, wherein the medicament is administered to the patient about every 3 weeks (Q3W) for about 4 cycles.

63: The use of any one of claims 60-62, wherein the anti-PD-L1 antibody or antigen binding fragment thereof is durvalumab.

64: The use of any one of claims 60-63, wherein the medicament comprises about 500-2000 mg of durvalumab.

65: The use of claim 64, wherein the medicament comprises about 1500 mg of durvalumab.

66: The use of any one of claims 61-63, wherein the medicament comprises about 20 mg/kg of durvalumab.

67: The use of any one of claims 60-69, further comprising an adjuvant therapy comprising an anti-PD1-L1 antibody or antigen binding fragment thereof, wherein the adjuvant therapy is administered to the patient after the patient receives a radical cystectomy.

68. The use of claim 67, wherein the adjuvant therapy comprises about 500-2000 mg of durvalumab.

69: The use of claim 68, wherein the adjuvant therapy comprises about 1500 mg of durvalumab.

70: The use of claim 69, wherein the adjuvant therapy comprises about 1500 mg of durvalumab administered to the patient about every 2 to 4 weeks (Q2W-Q4W) for up to about 12 cycles.

71: The use of claim 70, wherein the adjuvant therapy is administered to the patient about every 4 weeks (Q4W) for up to about 8 cycles.

72: The use of claim 67, wherein the adjuvant therapy comprises about 20 mg/kg of durvalumab administered to the patient about every 2 to 4 weeks (Q2W-Q4W) for up to about 12 cycles.

73: The use of claim 72, wherein the adjuvant therapy comprises about 20 mg/kg of durvalumab administered to the patient about every 3 weeks (Q2W-Q4W) for up to about 8 cycles.

74: Use of a combination therapy comprising about 1500 mg durvalumab and one or more platinum-based chemotherapies for the manufacture of a medicament for treating bladder cancer in a patient in need thereof before the patient receives a radical cystectomy;
wherein the medicament is administered to the patient about every 3 weeks (Q3W) for about 4 cycles; and
wherein an adjuvant therapy comprising about 1500 mg of durvalumab is administered to the patient about every 4 weeks (Q4W) for about 8 cycles following the radical cystectomy.

75: The use of any one of claims 60-74, wherein the one or more platinum-based chemotherapies is afatinib, bevacizumab, carboplatin, cetuximab, cisplatin, doxorubicin, emurafenib, erlotinib, gemcitabine, methotrexate, nedaplatin, oxaliplatin, paclitaxel, pemetrexed, and/or vinblastine.

76: The use of claim 75, wherein the one or more platinum-based chemotherapies are carboplatin and gemcitabine.

77: The use of any one of claims 60-76, wherein the one or more the platinum-based chemotherapies are (a) cisplatin administered to the patient at a dose of about 10-100 mg/m² Q3W; and (b) gemcitabine administered to the patient at a dose of about 500-1500 mg/m² Q3W.

78: The use of claim 77, wherein, if the patient has a creatinine clearance of about ≥ 60 mL/min, the one or more platinum-based chemotherapies are (a) cisplatin administered to the patient at a dose of about 70 mg/m² on day 1 of each cycle; and (b) gemcitabine administered to the patient at a dose of about 1000 mg/m² on days 1 and 8 of each cycle.

79: The use of claim 77, wherein if the patient has a creatinine clearance of about ≥ 40 mL/min to <60 mL/min, the one or more platinum-based chemotherapies are (a) cisplatin administered to the patient at a dose of about 35 mg/m² on days 1 and 8 of each cycle; and (b) gemcitabine administered to the patient at a dose of about 1000 mg/m² on days 1 and 8 of each cycle.

80: Use of a combination therapy comprising about 1500 mg durvalumab, gemcitabine, and cisplatin for the manufacture of a medicament for treating bladder cancer in a patient in need thereof before the patient receives a radical cystectomy;
wherein the medicament is administered to the patient about every 3 weeks (Q3W) for 4 cycles;
wherein gemcitabine is administered to the patient at a dose of about 1000 mg/m² on days 1 and 8 of each cycle;
wherein cisplatin is administered to the patient at a dose of about 70 mg/m² on day 1 of each cycle if the patient has a creatinine clearance of about ≥60 mL/min or at a dose of about 35 mg/m² on days 1 and 8 of each cycle if the patient has a creatinine clearance of about ≥40 mL/min to <60 mL/min; and
wherein an adjuvant therapy comprising about 1500 mg of durvalumab is administered to the patient about every 4 weeks (Q4W) for up to 8 cycles following the radical cystectomy.

81: The use of any one of claims 60-80, wherein the anti-PD-L1 antibody or antigen binding fragment thereof is administered to the patient prior to administration of the one or more platinum-based chemotherapies.

82: The use of any one of claims 60-80, wherein the anti-PD-L1 antibody or antigen binding fragment thereof is administered to the patient at the same time as administration of the one or more platinum-based chemotherapies.

83: The use of any one of claims 60-82, wherein the patient receives a radical cystectomy about 14 days to about 70 days following administration of 4 cycles of the anti-PD-L1 antibody or antigen binding fragment thereof and one or more platinum-based chemotherapies.

84: The use of claim 83, wherein the patient receives a radical cystectomy about 56 days following administration of 4 cycles of the anti-PD-L1 antibody or antigen binding fragment thereof and one or more platinum-based chemotherapies.

85: The use of any one of claims 67-70, wherein the adjuvant therapy comprises about 1500 mg of durvalumab administered to the patient every 4 weeks (Q4W) for up to about 8 cycles.

86: The use of any one of claim 67-70, 71-74, or 85 wherein the adjuvant therapy is administered to the patient about 28 days to about 150 days after the patient receives a radical cystectomy.

87: The use of claim 86, wherein the adjuvant therapy is administered to the patient about 42 days to about 120 days after the patient receives a radical cystectomy.

88: The use of any one of claims 60-87, wherein the bladder cancer is muscle-invasive bladder cancer (MIBC) or transitional cell carcinoma (TCC) of the urothelium.

89: The use of claim 88, wherein the MIBC is resectable MIBC.

90: The use of any one of claims 60-89 wherein the patient has not received prior systemic chemotherapy or immunotherapy for treatment of MIBC.


SEQUENCE LISTING (8 sequences)

SEQ ID NO: 1 - protein, 108 aa, synthetic construct
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 2 - protein, 12 aa, synthetic construct
RASQRVSSSYLA

SEQ ID NO: 3 - protein, 7 aa, synthetic construct
DASSRAT

SEQ ID NO: 4 - protein, 9 aa, synthetic construct
QQYGSLPWT

SEQ ID NO: 5 - protein, 121 aa, synthetic construct
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 6 - protein, 10 aa, synthetic construct
GFTFSRYWMS

SEQ ID NO: 7 - protein, 17 aa, synthetic construct
NIKQDGSEKYYVDSVKG

SEQ ID NO: 8 - protein, 12 aa, synthetic construct
EGGWFGELAFDY